(19) United States
(12) Patent Application Publication
(10) Pub. No.: US 2014/0274800 A1
(43) Pub. Date: Sep. 18, 2014

(54) TRANSCRIPTION FACTOR DECOYS FOR THE TREATMENT AND PREVENTION OF INFECTIONS CAUSED BY BACTERIA INCLUDING CLOSTRIDIUM DIFFICILE

(71) Applicant: PROCARTA BIOSYSTEMS LTD., Norwich (GB)

(72) Inventor: Michael MCARTHUR, Norwich (GB)

(73) Assignee: Procarta Biosystems Ltd., Norwich (GB)

(21) Appl. No.: 13/802,103

(22) Filed: Mar. 13, 2013

(51) Int. Cl. C12N 15/113

(52) U.S. Cl. CPC C12N 15/113
USPC 506/14; 536/24.1; 435/320.1

(57) ABSTRACT

Decoy nucleic acid sequences comprising a binding site for a target transcription factor, wherein the binding site is not operably linked to a gene, and wherein the transcription factor comprises a regulator of expression of a gene or genes in Clostridium difficile encoding one or more of: (i) a cellular growth factor; (ii) a cellular toxin; (iii) a cellular sporulation factor; (iv) a cellular stress response; and/or (v) a cellular essential gene are described. Uses of the decoys in antibacterial complexes for the treatment of bacterial infections are also described.

### FIELD OF THE INVENTION

The present invention relates to decoy nucleic acids sequences particularly those active against Clostridium difficile.

### BACKGROUND OF THE INVENTION

Clostridium difficile is an anaerobic, spore-forming bacterium that is able to colonise the human gut, with the resultant infection causing a wide range of symptoms from mild to severe diarrhoea, blood-stained stools, abdominal cramps caused by inflammatory response, fever that in severe cases can be fatal (Johnson (2009) J. Infection 58: 403-410). The incidence and severity of infection are strongly correlated with the extent and duration of antibiotic therapy that patients have previously received. This is because most antibiotics used to treat the gut are Broad-spectrum and will kill pathogenic and non-pathogenic bacteria alike. The effect of this sterilisation is that dormant spores of the commensal C. difficile are able to germinate and rapidly colonise the gut. These spores are not susceptible to antibiotic therapy or to a wide range of other methods used to disinfect surfaces of bacteria (e.g. heat, UV and various biocides). As the C. difficile infection proceeds, the bacteria begin to produce toxins (Toxin A and B) that cause a strong inflammatory response, leading to Pseudomembranous colitis (PMC) and giving rise to the symptoms associated with the infection (Bartlett et al. (1977) J. Infect. Dis. 136: 701-705; McDonald et al. (2005) N. Eng. J. Med. 353: 2433-2441). In the later stages of the infection, the bacteria once again produce endospores which are shed, along with live bacteria, in the faeces and greatly increase the chance of re-infection of the patient or spread to fellow patients. Hence, the recurrence rate of the disease is estimated to be between 20 and 45% following resolution of initial treatment (Aslam et al. (2005) Lancet Infect. Dis. 5: 549-557; McFarland et al. (2002) Am. J. Gasteroenterol. 97: 1769-1775) and the ability to break the cycle of re-infection is a major unmet medical need.

C. difficile naturally occurs in the gut microflora of newborns, a minority of the adult population below 65, and the majority of those over 65. Hence, C. difficile-associated disease (CDAD) is also correlated with elderly patients, especially those resident in nursing homes or hospitals, as well as all patients who have undergone gastrointestinal surgery or are immuno-compromised. As such C. difficile is a major cause of nosocomial infections and, in the UK, is the single largest source of infection: in 2003, twice as many deaths were attributable to CDAD as MRSA (Kuijper et al. (2006) Clin. Microbiol. Infection 12 (Supplement 6): 2-18), and [symbol] 3 billion was spent on treating CDAD across the EU as a whole. In the US, the annual costs associated with CDAD are estimated as $3.2 billion (O'Brien et al. (2007) Infect. Control Hosp. Epidemol. 28: 1217-1219-1227). An estimated additional [symbol] 7147 is spent per CDAD patient in the EU, compared to the cost of treating a non-CDAD patient (Vonberg et al. (2008) J. Hosp. Infect. 70: 15-20), which equates to an estimated additional annual spend of [symbol] 350 million in the UK where 5 of every 1000 patient days are attributable to CDAD (Bauer et al. (2011) Lancet 377: 63-73).

Globally, the incidence and severity of CDAD is increasing (Dubberke et al. (2010) Infect. Control Hosp. Epidemol. 10: 1030-1037). This can be linked to the emergence of new types of hyper-virulent strains, most notably PCR-ribotype 027, that overproduces Toxins A and B and induces severe diarrhoea (Warny et al. (2005) Lancet 366: 1079-1084) and results in increased rates of mortality (Pepin et al. (2005) CMAJ 173: 1037-1042). The PCR-ribotype 027 strain was first recognised as causing an outbreak in the Stoke Mandeville Hospital (UK) where it was responsible for 174 cases of CDAD and 19 (11%) deaths (Anon. (2005) CDR Weekly 15: 24). Within 2 years, the strain had been detected in most EU countries and North America (Freeman et al. (2010) Clin. Microbiol. Rev. 23: 529-549). Currently there are ten commonly recognised PCR ribotypes of C. difficile that have been implicated in hospital outbreaks, all with differing antibiotic sensitivities and virulence (Huang et al. (2009) Int. J. Antimicrob. Agents 34: 516-522). A particular challenge is ribotypes that show resistance to fluoroquinolines, such as 027 (McDonald et al. (2005) N. Eng. J. Med. 353: 2433-2441). Further, the type of PCR-ribotypes causing infections can be different between hospitals in the same country and change quickly: in the EU, as the incidence of the hyper-virulent strain wanes (PCR-ribotype 027) another one waxes (PCR-ribotype 078) (O'Donoghue & Kyne (2011) Curr. Opin. Gastroenterol. 27: 38-47). This emphasises the rapidity with which the epidemiology of CDAD can change and the extent of the challenge to develop robust and effective therapeutics and treatment strategies to respond to the varied threat of C. difficile infection.

Comparative genomics of clinical strains has revealed a high proportion of mobile genetic elements (Sebaihia et al. (2006) Nat. Genet. 38: 779-786) and a surprising lack of conservation between strains: only 85% of the genomes were shared between the genomic reference strain C. difficile 630 and the PCR-ribotype 027 strain QCD-32g58 (Scaria et al. (2010) PLOS One 12: e15147) and only 19.7% of genes were conserved in a panel of seventy five isolates (Stabler et al. (2006) J. Bac. 188: 7297-7305). This suggests that the ‘core gene set’ is particularly small, perhaps five hundred and fifty genes, thus limiting the number of therapeutic targets that will occur in all virulent strains (Janvilisri et al. (2009) J. Bac. 191: 3881-3891). In addition, there is little gene conservation between C. difficile and other closely related Clostridia species (Scaria et al. (2008) Mol. Cell. Probes 22: 238-243). These results underpin the observation that C. difficile is likely to be a highly adaptive pathogen, able to recruit by horizontal gene transfer antibiotic resistance mechanisms and novel virulence factors.

The antibacterials Clindamycin and Vancomycin that are commonly used to treat gut infections are effective against vegetative C. difficile cells but have no or little activity against their highly resistant endospores. Hence, once the antibiotic treatment has greatly reduced the microbial titre of the gut, C. difficile spores germinate to colonise the gut. Hence, there is a need for new treatments.

### BRIEF SUMMARY OF THE INVENTION

The present invention encompasses decoy nucleic acid sequences and antibacterial compositions, particularly decoy sequences and compositions active against Clostridium difficile. The invention particularly encompasses a decoy nucleic acid sequence comprising a binding site for a target transcription factor, wherein the binding site is not operably linked to a gene, and wherein the transcription factor comprises a regulator of expression of a gene or genes in Clostridium difficile encoding one or more of: (i) a cellular growth factor; (ii) a cellular toxin; (iii) a cellular sporulation factor; (iv) a cellular stress response; or (v) a cellular essential gene.

Some specific exemplary embodiments include, but are not limited to the following (note that the following embodiments are presented in claim-like format but do not supersede the claims or limit the scope of the claimed invention:

1. A decoy nucleic acid sequence comprising a binding site for a target transcription factor, wherein the binding site is not operably linked to a gene, and wherein the transcription factor comprises a regulator of expression of a gene or genes in Clostridium difficile encoding one or more of:
(i) a cellular growth factor;
(ii) a cellular toxin;
(iii) a cellular sporulation factor;
(iv) a cellular stress response; or
(v) a cellular essential gene.
2. A decoy nucleic acid sequence as claimed in claim 1, wherein the target transcription factor comprises a regulator of expression of one or more of:
(a) a gene or genes encoding cellular efflux pump protein, protein determining cell wall composition, cell wall density or cell wall metabolism;
(b) a cellular stress response gene or genes;
(c) a gene or genes encoding spores;
(d) a gene or genes encoding a toxin;
(e) an essential gene or genes.
3. A decoy nucleic acid sequence as claimed in claim 1, wherein the target transcription factor is selected from: Spo0A, TcdD, FapR and SigH.
4. A decoy nucleic acid sequence as claimed in claim 1, wherein the decoy nucleic acid sequence comprises: circular double stranded DNA or a linear oligonucleotide; and/or at least one element of secondary structure; and/or more than one copy of the transcription factor binding site; and/or additional sequence to the binding site(s); and/or modified bases or sugars to increase nuclease resistance of the nucleic acid sequence; and/or a plasmid or plasmid library.
5. A decoy nucleic acid sequence as claimed in claim 1, wherein the decoy nucleic acid sequence comprises multiple direct repeats of the transcription factor binding site.
6. A decoy nucleic acid sequence as claimed in claim 1, wherein the decoy nucleic acid sequence comprises multiple transcription factor binding sites.
7. A decoy nucleic acid sequence as claimed in claim 1, wherein the decoy nucleic acid sequence comprises a circular dumbbell.
8. A decoy nucleic acid sequence as claimed in claim 1, wherein the decoy nucleic acid sequence comprises a plasmid and wherein the plasmid has one or more copies of a monomer sequence comprising a snare sequence, the snare sequence comprising at least one transcription factor binding site wherein the binding site is not operably linked to a gene.
9. A decoy nucleic acid sequence according to any of the preceding claims, wherein the transcription factor binding site in the decoy nucleic acid sequence comprises a sequence selected from the group comprising:

(SEQ ID NO: 9)
TAC GAC A
(SEQ ID NO: 10)
TTT ACA NNN NNN NNN NNN NNN NNC TTT TT
(SEQ ID NO: 11)
TAG AAT TAG TAC CTG ATA CTA ATA ATT
(SEQ ID NO: 12)
GAA GGA ATA NNN NNN NNN NNA TAG AAT

10. A decoy nucleic acid sequence according to any of the preceding claims, wherein the decoy sequence is selected from: SEQ ID NOS: 13-14, 15-16, 17-18 and/or 19-20.
11. An antibacterial complex comprising one or more decoy nucleic acid sequences as claimed in claim 1 and one or more delivery moieties.
12. An antibacterial complex as claimed in claim 11, wherein one or more delivery moiety is selected from quaternary amine compounds and bis-aminoalkanes and unsaturated derivatives thereof, wherein the amino component of the aminoalkane is an amino group forming part of a heterocyclic ring.
13. An antibacterial complex as claimed in claim 11, wherein one or more delivery moiety is a quaternary derivative of quinoline or acridine.
14. An antibacterial complex as claimed in claim 11, wherein one or more delivery moiety is a bis-quinolinium
15. An antibacterial complex as claimed in claim 11, wherein one or more delivery moiety is dequalinium or an analogue thereof.
16. An antibacterial complex as claimed in claim 15, wherein the alkyl chain of the dequalinium analogue has between 8 and 14 methyl groups, or has 10 or 12 methyl groups.
17. An antibacterial complex as claimed in claim 16, wherein the dequalinium analogue is 10,10′-(dodecane-1,12-diyl)bis(9-amino-1,2,3,4-tetrahydroacridinium)dichloride.
18. Use of a decoy nucleic acid sequence as claimed in claim 1, or an antibacterial complex as claimed in claim 11, for the treatment of Clostridium difficile infection, optionally in combination with one or more antibiotics and/or other antibacterial agent(s).
19. A pharmaceutical composition or medicament comprising a decoy nucleic acid sequence as claimed in claim 1, or an antibacterial complex as claimed in claim 11, and a physiologically acceptable carrier or excipient, optionally in combination with one or more antibiotics and/or antibacterial agents.
20. A pharmaceutical composition or medicament as claimed in claim 19, for use in the treatment of Clostridium difficile infection.
21. A method of treating Clostridium difficile infection comprising administering a decoy nucleic acid sequence as defined in claim 1, or an antibacterial complex as claimed in claim 11, optionally in combination with one or more antibiotics and/or antibacterial agents.
22. An ex vivo method of killing Clostridium difficile, inhibiting Clostridium difficile growth, reducing Clostridium difficile sporulation, or reducing Clostridium difficile toxin production, the method comprising applying a decoy nucleic acid sequence as defined in claim 1, or an antibacterial complex as claimed in claim 11, optionally in combination with one or more antibiotics and/or antibacterial agents.
23. A cleaning composition comprising a decoy nucleic acid sequence as claimed in claim 1, or an antibacterial complex as claimed in claim 11, optionally in combination with one or more antibiotics and/or antibacterial agents.
24. A kit comprising one or more decoy nucleic acid sequences as claimed in claim 1, or one or more antibacterial complexes as claimed in claim 11, wherein the or each decoy is for use in killing, inhibiting growth, reducing sporulation or reducing toxin production in Clostridium difficile, the kit optionally further comprising one or more antibiotics and/or antibacterial agents.

### General Representations Concerning the Disclosure

This specification incorporates by reference to the fullest extent allowable by law all documents referred to herein and all documents filed concurrently with this specification or filed previously in connection with this application, including but not limited to such documents which are open to public inspection with this specification. Additionally this specification incorporates by reference to the fullest extent allowable by law the following US applications to the identical inventor: Ser. No. 12/681,588 filed 20 Aug. 2010; Ser. No. 13/062,922 filed 8 Mar. 2011; Ser. No. 13/578,625 filed 12 Aug. 2012; and Ser. No. 13/638,764 filed 1 Oct. 2012.

### DEFINITIONS

The terms “amino acid” and “amino acid sequence” refer to an oligopeptide, peptide, polypeptide, or protein sequence, or a fragment of any of these.

“Amplification” relates to the production of additional copies of a nucleic acid sequence e.g., using polymerase chain reaction (PCR).

The term “antibody” refers to intact immunoglobulin molecules as well as to fragments thereof, such as Fab, F(ab′)₂, and Fv fragments, which are capable of binding an epitopic determinant.

The term “similarity” refers to a degree of complementarily. There may be partial similarity or complete similarity. The word “identity” may substitute for the word “similarity.” A partially complementary sequence that at least partially inhibits an identical sequence from hybridizing to a target nucleic acid is referred to as “substantially similar.”

The phrase “percent identity” as applied to polynucleotide or polypeptide sequences refers to the percentage of residue matches between at least two sequences aligned using a standardized algorithm such as any of the BLAST suite of programs (e.g., blast, blastp, blastx, nucleotide blast and protein blast) using, for example, default parameters. BLAST tools are very commonly used and are available on the NCBI web site.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool set at default parameters. Such a pair of polypeptides may show, for example, at least 50%, at least 60%, at least 70%, at least 80%, at least 86%, at least 90%, at least 95%, or at least 98% or greater sequence identity over a certain defined length of one of the polypeptides.

The term “antisense” refers to any composition containing a nucleic acid sequence which is complementary to the “sense” strand of a specific nucleic acid sequence. Antisense molecules may be produced by any method including synthesis or transcription. Once introduced into a cell, the complementary nucleotides combine with natural sequences produced by the cell to form duplexes and to block either transcription or translation. The designation “negative” or “minus” can refer to the antisense strand, and the designation “positive” or “plus” can refer to the sense strand.

The terms “complementary” and “complementarity” refer to the natural binding of polynucleotides by base pairing. For example, the sequence “5′ A-G-T 3′” bonds to the complementary sequence “3′ T-C-A 5′.” Complementarity between two single-stranded molecules may be “partial,” such that only some of the nucleic acids bind, or it may be “complete,” such that total complementarity exists between the single stranded molecules. The degree of complementarity between nucleic acid strands has significant effects on the efficiency and strength of the hybridization between the nucleic acid strands. This is of particular importance in amplification reactions, which depend upon binding between nucleic acid strands, and in the design and use of peptide nucleic acid (PNA) molecules.

“Hybridization” refers to the process by which a polynucleotide strand anneals with a complementary strand through base pairing under defined hybridization conditions. Specific hybridization is an indication that two nucleic acid sequences share a high degree of identity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml denatured salmon sperm DNA. Generally, stringency of hybridization is expressed, in part, with reference to the temperature under which the wash step is carried out. Generally, such wash temperatures are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook et al., 1989, Molecular Cloning: A Laboratory Manual, 2^{nd} ed., vol. 1-3, Cold Spring Harbor Press, Plainview N.Y.; specifically see volume 2, chapter 9. High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acid sequences by virtue of the formation of hydrogen bonds between complementary bases. A hybridization complex may be formed in solution (e.g., C₀t or R₀t analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

The words “insertion” and “addition” refer to changes in an amino acid or nucleotide sequence resulting in the addition of one or more amino acid residues or nucleotides, respectively.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide containing the epitope A, or the presence of free unlabelled A, in a reaction containing free labelled A and the antibody will reduce the amount of labelled A that binds to the antibody.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 90%, at least 95%, or at least 98% or greater sequence identity over a certain defined length of one of the polypeptides.

### BRIEF DESCRIPTION OF THE DRAWINGS

FIG. 1: Chemical structure of 10,10′-(decane-1,10-diyl)bis(9-amino-1,2,3,4-tetrahydroacridinium)dichloride.

FIG. 2: Chemical structure of 10,10′-(dodecane-1,12-diyl)bis(9-amino-1,2,3,4-tetrahydroacridinium)dichloride.

FIG. 3: Nanoparticulate Spo0A TFD blocks sporulation in C. difficile.

FIG. 4: Cell growth of C. difficile cells treated with nanoparticles (NP) or untreated.

FIG. 5: Relative toxin production of C. difficile cells treated with nanoparticles or untreated over time.

FIG. 6: C. difficile cells treated with nanoparticulate FapR TFD prevent cell growth.

FIG. 7: Nanoparticles loaded with SigH TFD have a more antibacterial activity than empty.

FIG. 8: SigH TFD blocks early sporulation in C. difficile.

### DETAILED DESCRIPTION OF THE INVENTION

As discussed above, there is a need for new treatments for C. difficile.

In particular, a new treatment needs to have the following properties:
1. good efficacy against multiple ribotypes of C. difficile, including hyper-virulent strains;
2. specificity to prevent imbalancing gut microflora by killing commensal bacteria;
3. prevention of toxin production to suppress the inflammatory response leading to PMC;
4. inhibition of sporulation to decrease the likelihood of recurrence.

Transcription factor decoys (TFDs) have these attributes as they can be targeted to inhibit genetic pathways that are specific to C. difficile and involved in growth of the bacterium (essential genes), toxin production and sporulation, as well as combinations of all three.

TFDs are nucleic acids that contain the binding site for a transcription factor. When introduced into cells, they act as competitive inhibitors for the binding of the transcription factor to its genomic target and so modify the regulation of a targeted gene. In a therapeutic context, the targeted gene occurs in pathogenic bacteria and is essential for growth, environmental adaptation or onset of pathogenicity.

TFDs have been previously developed to treat a number of pathogens, most notably MRSA sepsis infections and to prevent growth of the Gram-negative Pseudomonas aeruginosa in vitro. A transcription factor decoy (TFD) is an oligonucleotide designed to bind to and sequester targeted bacterial transcription factors to prevent the expression of essential genes and so prevent growth.

It is against this background that the present invention has been devised. In particular, the present invention resides in a decoy nucleic acid sequence comprising a binding site for a target transcription factor, wherein the binding site is not operably linked to a gene, and wherein the transcription factor comprises a regulator of expression of a gene or genes in C. difficile encoding one or more of:
(i) a cellular growth factor;
(ii) a cellular toxin;
(iii) a cellular sporulation factor;
(iv) a cellular stress response; or
(v) a cellular essential gene.

The decoy sequence of the present invention is generally termed a Transcription Factor Decoy (TFD).

There are several developments required to adapt TFDs to treat C. difficile infections. These include:
1. identifying TFD sequences to prevent growth of C. difficile;
2. identifying TFD sequences to block toxin production to suppress inflammatory response and other virulence factors;
3. identifying TFD sequences to prevent sporulation and so lessen the extent of recurrence;
4. oral formulation for delivery to the gut lumen where the infection is located.
   Thus, the present invention also resides in a decoy nucleic acid sequence wherein the target transcription factor comprises a regulator of expression of one or more of:
(a) a gene or genes encoding cellular efflux pump protein, protein determining cell wall composition, cell wall density or cell wall metabolism;
(b) a cellular stress response gene or genes;
(c) a gene or genes encoding spores;
(d) a gene or genes encoding a toxin;
(e) an essential gene or genes.

Preferably, the target transcription factor is selected from: Spo0A, TcdD, FapR and SigH.

Genetic manipulation of C. difficile is notoriously difficult and, although there are a few examples of C. difficile knockouts (e.g. Heap et al (2007) J. Microbiological Methods 70: 452-464), it is only recently that effective tools to disrupt genes, complement the mutations and stably introduce plasmids have been developed for C. difficile (Bouillaut et al (2011) Curr. Protoc. Microbiol. Chapter 9: Unit 9A2). Hence, it has not been possible to identify essential genes using insertional mutagenesis or validate candidate genes by marking targeted knock-outs or knock-ins, as has been achieved for other pathogenic bacteria. Therefore, candidate essential genes must be identified on the basis of homology or orthology with characterised genes from other bacteria (such as the essential genes identified in Bacillus subtilis (Kobayashi et al (2003) Proc. Natl. Acad. Sci. USA 100: 4678-4683)), from inferred function, from patterns of expression in vitro (Karlsson et al (2008) Microbiology 154: 3430-3436) and in vivo (Scaria et al (2011) J. Infect. Dis. 203: 1613-1620 and by comparative genomic analysis of pathogenic and hyper-virulent strains (Scaria et al (2010) PLOS One 12: e15147).

Gene-essentiality studies have been performed for numerous bacteria and tested with multiple conditions. Although such datasets are not available for C. difficile, it is anticipated that some of the essential genes identified in a closely related bacteria or common to all bacteria (a ‘minimal gene set’) will be useful in identifying candidate essential genes in this organism (Gerdes et al (2006) Curr. Opin. Biotech. 17: 448-456). This may be on the basis of sequence or functional homology. The latter is becoming increasingly important as new system biology tools are being developed to identify seemingly disparate genes in separate organisms that control similar metabolic pathways or functional modules that have been identified as essential (Overbeek et al (2005) Nucl. Acids Res. 33: 5691-5702).

B. subtilis is one of the best studied bacteria and is a model organism for the low-G+C Gram-positive bacteria that includes other Firmicutes such as C. difficile. Like C. difficile, B. subtilis can grow anaerobically and also sporulates, apparently sharing many of the regulatory machinery controlling the switch to sporulation (Brown et al (1994) Mol. Micro. 14: 411-426). Hence, homologues of essential genes from B. subtilis may be similarly important in C. difficile and constitute targets for TFD therapeutics. Essential genes have been identified in B. subtilis by sequentially knocking out each gene to determine which are necessary for growth (Kobayashi et al (2003) Proc. Natl. Acad. Sci. USA 100: 4678-4683). Of the approximately four thousand one hundred genes tested in this study and incorporating previously identified genes, these researchers predicted that two hundred and seventy one genes were essential (6.6% of the total) as listed in Supplementary Table 4 associated with Kobayashi et al (supra). The most likely targets for TFD therapeutics would be those essential genetic pathways involved in primary metabolism or maintaining the cell envelope. These include:
1. fatty acid biosynthesis necessary for synthesis of membrane lipids (accA, accB, accC, accD, acpA, acpS, birA, fabD, fabF, plsX and fabG);
2. phospholipid biosynthesis necessary for synthesis of membrane lipids;
3. peptidoglycan biosynthesis;
4. diaminopimelate biosynthesis;
5. isoprenoid biosynthesis;
6. menquinone biosynthesis;
7. synthesis of co-factors such as acetyl-coenzyme A, folate, S-adenosylmethionine, Fe-sulfate clusters, Nicotinamide adenine dinucleotide (NAD);
8. Na/H transporters (mrp genes).

Additional candidates have been identified in B. subtilis by examining pairs of genes, it being argued that the duplicated gene may compensate for the loss of function of the first gene in the pair and so such genes would be missed in conventional screens for essentiality. Studies have identified a further set of candidate genes and those with homologues in C. difficile are shown in Table 1 below.

Two C. difficile toxins are considered to be key virulence factors and the causative agents of antibiotic-associated PMC in patients suffering from CDAD (Keuhne et al (2010) Nature Letters 467: 711-714). The mechanism of action of the toxin A, an enterotoxin, and the cytotoxin, toxin B, involve catalysing the glycosylation of Rho-GTPases, inhibiting their regulatory function on eukaryotic actin cytoskeletal components (Just et al (1995), Nature 375: 500-503). This results in fluid secretion leading to diarrhoea, mucosal damage and colon inflammation (Kelly et al (1994) New Eng. J. Med. 330: 257-262). These toxins are expressed during late exponential/stationary phase, in conjunction with the onset of sporulation. The genes encoding the toxins, tcdA and tcdB, are contained within the 19.6 kilo-base (kb) pathogenecity locus (PaLoc) that includes a gene encoding a positive transcriptional regulator, tcdD. The gene product from which TcdD (but also previously referred to as TcdR or ToxR) presents is an attractive target for TFD technology: TcdD upregulates expression of its own gene (Mani et al (2002), J. Bac, 184: 5971-5978) and tcdA and tcdB as the cells enter stationary phase (Mani and Dupuy (2001), PNAS, 98: 5844-5849) as well as responding to various environmental stimuli, including mammalian host body temperature (Karlsson et al (2003), Infection and Immunity, 71: 1784-1793).

Strains of C. difficile, such as CD37, that completely lack the PaLoc gene cluster are avirulent (Mani et al (2002), J. Bac, 184: 5971-5978), suggestive that TFD-mediated down-regulation of the tcdA and tcdB genes may alleviate the symptoms in CDAD patients. The binding site for the TcdD has been described and the four incidences of the binding sites for TcdD within the promoters of PaLoc are shown below (Dupuy & Matamouros (2006) Res. Microbiol. 157: 201-205), along with a derived consensus sequence.

(SEQ ID NO: 1)

| | |
|---|---|
| Consensus | 5′ AAGTTTACAAAATTATATTAGAATAACTTTTTATT |
| sequence: | |

(SEQ ID NO: 2)

| | |
|---|---|
| PtcdA: | 5′ ATGTTTACAAATTACTATCAGACAATCTCCTTATC |

(SEQ ID NO: 3)

| | |
|---|---|
| PtcdB: | 5′ AAGTTTACATATTTATTTCAGACAACGTCTTTATT |

(SEQ ID NO: 4)

| | |
|---|---|
| P1tcdD: | 5′ AAGTTTACATAATTATTTAATAATTATGTAATTGT |

(SEQ ID NO: 5)

| | |
|---|---|
| P2tcdD: | 5′ GTTTTTACAATACTTTATTAATATAAAGTTTATTG |

The ability of C. difficile to produce resistant and highly infectious spores is likely to be one of the major reasons for the high rate of recurrence of C. difficile infections. A C. difficile strain with deletion of Spo0A shows markedly lower levels of re-infection and transmission than the wild-type control in mouse models (Deakin et al (2012) Infection Immunity 80: 2704-2711). Spo0A is a highly conserved transcriptional regulator that plays a key role in initiating sporulation in Bacillus and Clostridium species (Escobar & Castano (2009) In silico Biol. 9: 149-162). Spo0A has been extensively studied in B. subtilis and the Spo0A regulon, the environmental signals that control its activity, has been described and importantly the sequence to which it binds, the Spo0A box: TGNCGAA (SEQ ID NO:6; Molle et al (2003) Mol. Micro. 50: 1683-1701). In C. difficile, Spo0A also has a role in toxin production and the severity of other virulence factors (Underwood et al (2009) J. Bac. 191: 7296-7305). Unusually the binding site appears to be conserved between Bacilus and Clostridium species (Ravagnani et al (2000) Mol. Micro. 37: 1172-1185). The Spo0A box occurs in several C. difficile promoters: spo0A itself, ptb, abrB, spollAA, CD2492 and sigH (Saujet et al (2011) J. Bac. 193: 3186-3196). A single SpoA box occurs in its own promoter on the basis of similarity to the consensus and is of the sequence: TGTCGAA (SEQ ID NO:7).

Bacterial gene expression is controlled primarily at the level of transcription by the recognition of promoters by the RNA polymerase. This interaction is directed by a diverse family of proteins called the sigma factors. These recruit the RNA polymerase to the promoters of certain genes following recognition of their binding sites. The most common site, which mostly is found upstream of constitutively expressed ‘housekeeping’ genes, is the ‘−35/−10’ site (as the binding sites are centred 10 and 35 nucleotides upstream from the transcriptional start site) which is recognised by the ubiquitous sigma-70 protein, typically encoded by the gene sigA. However, in most bacteria, including the Clostridia, a large number of alternative sigma-70 factors exist that recognise different consensus sites to induce distinct regulons (Helmann (2002) Adv. Microb. Physiol. 46: 47-110). Bacteria seem to use these factors to control sets of genes required to respond to environmental signals, for example cell wall damage (Ho & Ellermeier (2012) Curr. Opin. MicrobioL 15: 182-188), or developmentally regulated processes which can include the onset of pathogenicity (Bashyam & Hasnain (2004) Infect. Evol. 4: 301-308). Hence, alternative sigma-factors are good targets for TFDs as they meet several of the criteria of development of a useful therapeutic. For example, TFD-mediated inhibition of the regulon of alternative sigma factors would affect multiple genes, including those with essential functions that will prevent cell growth. Additionally the alternative sigma factors often positively regulate their own transcription so that, by blocking their function, TFD treatment would prevent these factors from becoming highly expressed, helping to inhibit induction of the regulon. Finally the alternative sigma factors are specific to bacteria and so TFDs designed to inhibit them will not interfere with host transcriptional machinery. In C. difficile there are nineteen putative sigma-70 factors (Table 1) that have been identified, largely on the basis of homology to known proteins but some by functional characterisation and more may yet be found.

TABLE 1
Putative Sigma factors in C. difficile

| Locus | Name | Type | Comments |
|---|---|---|---|
| CD0011 | sigB | 70 | Implicated in stress response and regulation of |
| | | | bile resistance genes (Sebaihia et al. (2006) |
| | | | Nat. Genet. 38: 779-786) |
| CD0057 | sigH | 70 | Key regulator of sporulation and cell growth |
| | | | (Saujet et al. (2011) J. Bac. 193: 3186-3196) |
| CD0266 | fliA | 70 (28)* | Homologue controls spore development in B. subtilis |
| | | | (Errington et al. (1996) Philos. Trans. |
| | | | Roy. Soc. Lond. B. Biol. Sci. 351: 537-542) and |
| | | | in P. aeruginosa the homologue controls |
| | | | flagellin biosynthesis (Starnbach & Lory (1992) |
| | | | Mol. Micro. 6: 459-469) |
| CD0359 | — | 70 | |
| CD0435 | — | 70 | |
| CD0659 | tcdD | 70 | Directs expression of toxin genes tcdA and tcdB |
| | | | (Karlsson et al. (2003) Infect. Immun. 71: |
| | | | 1784-1793) |
| CD0677 | csfT | ECF | Autoregulated, highly expressed in stationary |
| | | | phase and in response to lysozyme (Ho & |
| | | | Ellermeier (2011) Infect. Immun. 79: 3229-3238) |
| CD0772 | sigF | 70 | Homologue in C. perfringens regulates other |
| | | | sigma factors (sigE and sigK) and affects |
| | | | sporulation and toxin production (Li & McClane |
| | | | (2010) Infect. Immun. 78: 4286-4293) |
| CD1094 | — | 70 | |
| CD1230 | sigK | 70 | Positive regulator of sporulation (Haraldsen & |
| | | | Sonenshein (2003) Mol. Micro. 48: 811-821) |
| CD1455 | rpoD1/sigA1 | 70 | ‘Housekeeping’ sigma factor |
| CD1498 | rpoD2/sigA2 | 70 | ‘Housekeeping’ sigma factor |
| CD1558 | csfV/sigV | ECF | Autoregulated, highly expressed in stationary |
| | | | phase and in response to lysozyme (Ho & |
| | | | Ellermeier (2011) Infect. Immun. 79: 3229-3238) |
| CD1887 | csfU | ECF | Autoregulated, highly expressed in stationary |
| | | | phase and in response to lysozyme (Ho & |
| | | | Ellermeier (2011) Infect. Immun. 79: 3229-3238) |
| CD2223 | — | 70 | |
| CD2642 | sigG | 70 | Homologue in C. perfringens affects sporulation |
| | | | (Li & McClane (2010) Infect. Immun. 78: |
| | | | 4286-4293) |
| CD2643 | sigE | 70 | Homologue in C. perfringens affects sporulation |
| | | | and toxin production (Li & McClane (2010) |
| | | | Infect. Immun. 78: 4286-4293) |
| CD3329 | — | 70 | |
| CD3371 | — | 70 | |

*Designated 28 as it is a class of truncated sigma-70 proteins

In many bacteria, including C. difficile, there is additionally a smaller class of structurally distinct sigma factors, known as sigma-54 or rpoN-like, that recognise a different consensus site centred around the −24/−12 position. Usually, each species contains only a single copy of a sigma-54 and, despite being initially discovered in Esherichia coli for its role in regulating nitrogen metabolism (hence rpoN) amongst other bacteria, there is no clear conservation of function, variously having roles in regulating energy metabolism, flagellation, response to bacteriophage and others (Buck et al (2000) J. Bac. 182: 4129-4136). In C. difficile, the gene encoding the putative sigma-54 factor is glnF (CD630_{—}31760) which has a high degree of homology to E. coli rpoN that controls, amongst other things, the essential processes of nitrogen assimilation and metabolism, for example by regulating the synthesis of glutamine (Reitz and Schneider (2001) Micro. Mol. Biol. Rev. 65: 422-444). However, it is currently not possible to identify homologues of the E. coli rpoN regulon in C. difficile, meaning that it is not possible to identify common motifs found in the promoters of these co-regulated genes which may candidates for the GlnF binding site, especially those similar to the sigma-54 consensus site 5′-TGGCAnnnnnnnTTGCw-3′ (SEQ ID NO:8; Barrios et al (1999) Nucl. Acids Res. 27: 4305-4313). Direct experimental methods will be needed to identify the glnF regulon, either relying on gene knockout approaches or in vitro transcription (MacLellan et al (2009) Methods 47: 73-77).

Two-component signal (TCS) transduction systems enable bacteria to sense, respond and adapt to changes in their environment or in their intracellular state. The systems consist of a sensory protein, usually incorporating a histidine kinase, and a response regulator that is the target for phopshorylation and, when activated, acts as a transcription factor to control a set of response genes. The systems make attractive, novel targets for antibacterial therapeutics (Stephenson and Hoch (2002) Curr. Opin. Pharmacol. 2: 507-512) and examples have been identified as essential genes (WalKR in Staphylococcus aureus and YycFG in B. subtilis). TFDs have been designed successfully to target the binding site for the WalKR TCS in S. aureus and this has been shown to have a potent antibacterial effect. TCS have been shown to mediate bacterial response to nutrient limitation, cell wall damage and quorum sensing, all of which play key roles in disease progression. Using pathway databases, such as the Kyoto Encyclopedia of Genes and Genomes (KEGG), it is possible to identify potential TCS systems in C. difficile that will likely affect cell survival and pathogenicity.

Once such a list has been compiled, it will be examined to determine whether or not the sequences constitute good targets for TFD-mediated regulation. To do so, the sequences should have some or all of the following characteristics:
1. not constitutively expressed;
2. positively regulated;
3. strongly induced;
4. part of a large operon.

The process of nanoparticulate delivery of the TFDs will cause stress to the bacteria, as has been shown for Staphylococcus aureus where a canonical Cell Wall Stress (CWS) Regulon has been defined (reviewed in Dengler et al (2011) BMC Microbiol. Epub 2011 Jan. 20). There is little literature on inducing the CWS Regulon in C. difficile. It has been shown that treatment with lysozyme induces a stress-response mediated by only one of the ECF sigma factors, sigF, but not the others (Ho & Ellermeier (2011) Infect. Immun. 79: 3229-3238). This suggests that it will be difficult to predict what transcription factors will mediate stress response to nanoparticulate delivery and so it will need to be determined empirically. Such an experiment will involve challenging the bacterium with a nanoparticle loaded with a scrambled TFD sequence that does not bind any transcription factors and comparing the transcriptomic response with an untreated bacterium with RNA-seq to identify those genes induced by nanoparticles.

Once a set of co-regulated target genes have been identified, it remains to identify DNA sequences implicit in their regulation, such as binding sites for transcription factor(s) controlling induction of the regulon. This can be achieved either by bioinformatical or empirical methods.

A suitable bioinformatical method may involve: assigning the set of genes to operons (this may involve discovery of new gene candidates); creating a set of sequences containing candidate promoters, defined as sequences 50 to 300 bp upstream of the first initiation codon of each member of the ‘operon set’; interrogation of the ‘promoter set’ to identify candidate binding motifs, including:
1. previously characterized binding motifs;
2. new motifs found to be common to the set of promoters or a subset;
3. sequences with secondary structure commonly associated with binding motifs, such as:
   a. direct repeats of short stretches of sequences (3 to 12 bp), which may be contiguous or separated by some distance;
   b. inverted repeats of short stretches of sequences;
   c. sequences with similarity to known RNA polymerase recognition sequences, such as variants of the typical −10/−35 sequences recognised by the family of sigma-70 proteins or the −24/−12 sequences recognised by the family of sigma-54 proteins;
   d. palindromic sequences;
   e. specialist secondary structures such as described in the literature and typified by riboswitches;
   f. those which have the potential to form further tertiary structures, some of which have been implicated in controlling transcriptional regulation, such as triplex or H-DNA.

Each step of the process may be informed by biological data. For example, transcriptomic data may be used to refine the operon set as both microarray and RNA sequencing data may help identify co-regulated genes. Further RNA sequencing data can identify the transcriptional start sites of genes and operons. Other types of experiments may also identify the start sites of promoters and these include PCR based methods (e.g. RACE PCR), Northern blotting and other well established methods.

Where a transcription factor has been identified as a good target for TFD-mediated control but the set of genes that it regulates is not known, other strategies to identify candidate TFD sequences may be employed. In most instances it can be predicted that the transcription factor will form a binary complex in vitro between DNA and protein, with protein binding either typically as a monomer or dimer. A well established approach would then be to produce and purify a recombinant version of the transcription factor and identify high affinity binding sequences by library of DNA fragments (potentially made by shearing the genome of the target organism) or to libraries of synthetic oligonucleotides. Various methods, both physical and biological, may be used to detect and quantify binding strength. Examples of such processes include those described in WO 2009/044154.

Where the transcription factor is a sigma factor, binding only occurs in the context of intact RNA polymerase, which consists of multiple proteins. Though possible, in vitro reconstitution of an intact and active RNA polymerase is challenging and makes detection of recognition sequences by an iterative selection procedure difficult. An alternative approach is to use reconstituted RNA polymerase to initiate transcription on genomic DNA and use microarray analysis or direct sequencing to map the sites of transcriptional initiation. This can be treated as a ‘promoter set’ for analysis as described above.

It will be appreciated that the decoy nucleotide acid sequence of the present invention may comprise: circular double stranded DNA or a linear oligonucleotide; and/or at least one element of secondary structure; and/or more than one copy of the transcription factor binding site; and/or additional sequence to the binding site(s); and/or modified bases or sugars to increase nuclease resistance of the polynucleotide; and/or a plasmid or plasmid library.

The decoy nucleic acid sequence may comprise multiple direct repeats of the transcription factor binding site and/or may comprise multiple transcription factor binding sites.

In a preferred embodiment, the decoy nucleic acid sequence comprises a circular dumbbell.

Optionally, the decoy nucleic acid sequence comprises a plasmid and wherein the plasmid has one or more copies of a monomer sequence comprising a snare sequence, the snare sequence comprising at least one transcription factor binding site wherein the binding site is not operably linked to a gene.

Preferably, the transcription factor binding site in the decoy nucleic acid sequence comprises a sequence selected from the group comprising:

(SEQ ID NO: 9)
TAC GAC A
(SEQ ID NO: 10)
TTT ACA NNN NNN NNN NNN NNN NNC TTT TT
(SEQ ID NO: 11)
TAG AAT TAG TAC CTG ATA CTA ATA ATT
(SEQ ID NO: 12)
GAA GGA ATA NNN NNN NNN NNA TAG AAT

Also preferred is a decoy sequence is selected from: SEQ ID NOS: 13-14, 15-16, 17-18 and/or 19-20.

The decoy nucleic acid sequence of the invention may be an oligonucleotide sequence or a polynucleotide sequence. An oligonucleotide sequence is generally recognised as a linear sequence of up to 20 nucleotides joined by phosphodiester bonds, while a polynucleotide sequence typically has more than 20 nucleotides and maybe single or double stranded with varying amounts of internal folding. The backbone may also be modified to incorporate synthetic chemistries known either to reduce the charge of the molecule or increase its stability in biological fluids. Examples of these include peptide nucleic acids (PNA), linked nucleic acids (LNA), morpholino oligonucleotides and phosphorothioate nucleotides and combinations of these.

The sequences provided herein illustrate single strands of the binding sites. However, it will be appreciated that in nature and in the TFDs of the present invention, the sequences will be double stranded. The complementary strands to the sequences listed herein are clearly and easily derivable, for example from Molecular Cloning: A Laboratory Manual (3^{rd} Edition), 2001 by Joseph Sambrook and David Russell.

Decoys of the invention may be prepared by any suitable method, such as those methods described in WO 2009/044154.

For example, dumbbells may be prepared as linear oligonucleotides and then ligated with T4 ligase. Alternatively dumbbell decoys may be prepared by PCR using appropriate primers. Each primer generally contains a portion which will form the stem loop of the dumbbell structure. PCR amplification using the primers is typically followed by restriction digest of the amplification product and ligation to form the closed circle dumbbell.

Alternatively, dumbbells may be prepared by restriction digest of a plasmid. Digestion is followed by ligation to form the closed circle dumbbell structure.

A decoy sequence may comprise a variant or analogue of a native or consensus binding sequence, which retains decoy function. A variant or analogue may be prepared by altering, for example, 1, 2, 3, 4, 5, 6, 7, 8, 9, 10 or more nucleotides in the parent sequence. For example, footprinting experiments may indicate that particular nucleotides are less crucial for transcription factor binding and might be altered.

A putative decoy sequence may be tested for ability to compete with a given transcription factor binding site by introducing a decoy polynucleotide comprising the decoy sequence into a suitable host cell. The host cell includes the target transcription factor binding site operably linked to a gene or genes whose expression can be determined directly or indirectly, e.g. by screening for a change in phenotype. Decoy function of a test sequence can be determined by screening for a change in expression of the gene(s) or a change in the phenotype. Methods for testing and researching suitable decoy sequences are described in WO 2009/044154.

In a second embodiment, the present invention resides in an antibacterial complex comprising one or more decoy nucleic acid sequences and one or more delivery moieties, wherein the one or more transcription factor comprises a regulator of expression of a gene or genes in Clostridium difficile encoding one or more of:
(i) a cellular growth factor;
(ii) a cellular toxin;
(iii) a cellular sporulation factor;
(iv) a cellular stress response; or
(v) a cellular essential gene.

It will be appreciated that an antibacterial complex may comprise one TFD complexed with a single delivery moiety, or two or more TFDs complexed with a single delivery moiety.

Ideally, the delivery moiety is, or is part of a nanoparticulate delivery system consisting of a quaternary amine compound or a bis-aminoalkane and unsaturated derivatives thereof. The term “aminoalkanes” as used herein refers to amino groups (preferably tertiary amino groups) that form part of a heterocyclic ring. The delivery moiety associates with the phosphate backbone of the TFD to shield the TFD's charge and protect it from degradation in biological fluids, whilst allowing passive transport across bacterial membranes to deliver the TFDs into the cytoplasm of the cell.

Exemplary of such compounds are compounds of the formula (I):

Q-(CH₂)ₚ-A-(CH₂)_{q}—R³ (I)

wherein Q is selected from:
(a) a group Q¹ having the formula:

[chemical structure]

and
(b) a group Q², Q²-NH—, Q²-O—, or Q²-S— wherein Q² is selected from monocyclic, bicyclic and tricyclic heteroaromatic groups of 5 to 14 ring members, of which 1, 2 or 3 are heteroatom ring members selected from N, O and S provided that at least one nitrogen ring member is present, wherein the heteroaromatic groups are optionally substituted by one or two substituents R^{4a} and wherein the said one nitrogen ring member may form an N-oxide or may be substituted with C₁₋₄ alkyl, phenyl-C₁₋₄ alkyl or di-phenyl-C₁₋₄ alkyl to form a quaternary group, wherein the phenyl moieties in each case are optionally substituted with one or two halogen, methyl or methoxy groups;
m is 0 or 1;
n is 0 or 1;
p and q are the same or different and each is an integer from 1 to 12;
A is a bond or is selected from a naphthalene, biphenyl, terphenyl, phenanthrene, fluorene, stilbene, a group C₆H₄(CH₂)ᵣC₆H₄, a group C₆H₄—C≡C—C₆H₄, a pyridine-2,6-diyl-bis(benzene-1,4-diyl) group, a group CH═CH—(CH₂)ₛ—(CH═CH)ₜ—; and a group C≡C—(CH₂)ᵤ—(C≡C)ᵥ—; wherein r is 0-4, s is 0 to 4, t is 0 or 1; u is 0-4 and v is 0 or 1;
when n is 1, R⁰, R¹ and R² are each selected from C₁₋₄ alkyl; and when n is 0, then N, R¹ and R² together form a monocyclic, bicyclic or tricyclic heteroaromatic group of 5 to 14 ring members, of which one is the nitrogen atom N and 0, 1 or 2 are further heteroatom ring members selected from N, O and S, and wherein the heteroaromatic group is optionally substituted by one or two substituents R^{4b}; and
R³ is selected from hydrogen, C₁₋₄ alkyl, halogen, monocyclic carbocyclic groups of 3 to 7 ring members each optionally substituted by one or two substituents R^{4c}, a group Q; a group —NH-Q², a group —O-Q² and a group —S-Q²; and
R^{4a}, R^{4b} and R^{4c} are the same or different and each is selected from C₁₋₄ alkyl optionally substituted with one or more fluorine atoms: C₁₋₄ alkoxy optionally substituted with one or more fluorine atoms; nitro; amino; mono- and di-C₁₋₄ alkylamino; halogen; phenyl-C₁₋₂ alkyl wherein the phenyl moiety is optionally substituted with one or two methoxy, methyl or halogen substituents; ureido and guanidinyl.

In one preferred embodiment, Q is a group Q¹:

[chemical structure]

Accordingly, one preferred sub-group of compounds within formula (I) is represented by formula (II):

[chemical structure]

wherein R¹, R², m, p, A, q and R³ are as defined in respect of formula (I).

One preferred sub-group of compounds within formula (II), wherein R³ is Q¹ and n in each instance is 0, can be represented by the formula (III):

[chemical structure]

wherein R¹, R², m, p, A and q are as defined in respect of formula (I).

In the compounds of formulae (I), (II) and (III), when m is 1, the nitrogen atom N must be a quaternary nitrogen. Accordingly, the compounds of formulae (I), (II) and (III) wherein m is 1 will comprise one or more anions as counter ions, for example anions derived from mineral acids, sulphonic acids and carboxylic acids.

When N, R¹ and R² together form a monocyclic, bicyclic or tricyclic heteroaromatic group of 5 to 14 ring members, typically the group contains either the nitrogen atom N as the sole heteroatom ring member or contains a second heteroatom ring member selected from N, O and S.

When N, R¹ and R² together form a monocyclic, bicyclic or tricyclic heteroaromatic group, the nitrogen atom N forms part of an aromatic ring. Preferred heteroaromatic groups are monocyclic aromatic rings; bicyclic heterocyclic rings in which both rings are aromatic; bicyclic heterocyclic rings in which one nitrogen-containing ring is aromatic and the other ring is non-aromatic; and tricyclic rings in which two rings, including a nitrogen-containing ring, are aromatic and the other ring is non-aromatic.

When N, R¹ and R² together form a monocyclic, bicyclic or tricyclic heterocyclic group of 5 to 14 ring members, the heterocyclic group is preferably selected from quinoline; isoquinoline; acridine; tetrahydroacridine and ring homologues thereof; pyridine; benzoimidazole; benzoxazole and benzothiazole. By ring homologues of tetrahydroacridine is meant compounds containing the core structure:

[chemical structure]

wherein y is 1 or 3. By tetrahydroacridine is meant a compound having the core structure above wherein y is 2.

In a preferred embodiment, the delivery moiety is a quaternary derivative of quinoline or acridine, in particular 1, 2, 3, 4-tetra-hydro-9-amino-acridine. Suitable derivatives of 1, 2, 3, 4-tetra-hydro-9-amino-acridine are described in U.S. Pat. No. 3,519,631, the contents of which are incorporated herein by reference. Of particular interest are the compounds and formulae exemplified in Examples 17 to 25 of U.S. Pat. No. 3,519,631 and analogues thereof and WO 2011/098829. Suitable quinoline derivatives are the bis-quinolinium compounds, such as dequalinium, and analogues thereof.

In a particularly preferred embodiment, the complex comprises a dequalinium analogue to enable the design of a dequalinium compound that has enhanced stability (both to dilution and the presence of salt) and yet has a similar or improved toxicity profiles to dequalinium. Such an analogue has been described that forms more stable complexes (Compound 7, Galanakis et al. (1995) J. Med. Chem. 35: 3536-3546) as tested by various physiochemical parameters such as ability to bind DNA to the exclusion of fluorescent dye SYBR-green, size of particles formed as measured by Dynamic Light Scattering and visualised with electron microscopy and their stability in elevated concentrations of salt and on dilution and storage for extended periods (Weissig et al. (2001) S. T. P. Pharma Sciences 11:91-96).

Examples of dequalinium and its analogues are compounds of the formula (IV):

[chemical structure]

wherein:
p and q are the same or different and each is an integer from 1 to 12;
A is a bond or is selected from naphthalene, biphenyl, terphenyl, phenanthrene, fluorene, stilbene, a group C₆H₄(CH₂)ᵣC₆H₄, a group C₆H₄—C≡C—C₆H₄, a pyridine-2,6-diyl-bis(benzene-1,4-diyl) group, a group CH═CH—(CH₂)ₛ—(CH═CH)ₜ—; and a group C≡C—(CH₂)ᵤ—(C≡C)ᵥ—; wherein r is 0-4, s is 0 to 4, t is 0 or 1; u is 0-4 and v is 0 or 1;
R⁸, R⁹ and R¹⁰ are the same or different and are each selected from hydrogen; C₁₋₄ alkyl optionally substituted with one or more fluorine atoms: C₁₋₄ alkoxy optionally substituted with one or more fluorine atoms; nitro; amino; mono- and di-C₁₋₄ alkylamino; halogen, phenyl-C₁₋₂ alkyl wherein the phenyl moiety is optionally substituted with one or two methoxy, methyl or halogen substituents; ureido and guanidinyl; or R⁹ and R¹⁰ link together to form an alkylene chain (CH₂)_{w} wherein w is 3 to 5; and R^{8a}, R^{9a} and R^{10a} are the same or different and are each selected from hydrogen; C₁₋₄ alkyl optionally substituted with one or more fluorine atoms: C₁₋₄ alkoxy optionally substituted with one or more fluorine atoms; nitro; amino; mono- and di-C₁₋₄ alkylamino; halogen, phenyl-C₁₋₂ alkyl wherein the phenyl moiety is optionally substituted with one or two methoxy, methyl or halogen substituents; ureido and guanidinyl; or R^{9a} and R^{10a} link together to form an alkylene chain (CH₂)_{w} wherein w is 3 to 5.

Within formula (IV), one subset of compounds is the subset in which A is a bond, a group CH═CH—(CH₂)ₛ—(CH═CH)ₜ—; or a group C≡C—(CH₂)ᵤ—(C≡C)ᵥ—. Within this sub-set, preferably A is a bond, i.e. there is a saturated alkylene chain extending between the nitrogen atoms of the two quinoline rings.

When A is a bond, typically the sum of p and q is in the range from 3 to 22, preferably in the range from 6 to 20, and more preferably from 8 to 18. Particular examples are compounds in which p+q=8, or p+q=9, or p+q=10, or p+q=11, or p+q=12, or p+q=13, or p+q=14, or p+q=15, or p+q=16, or p+q=17 or p+q=18.

In each of the foregoing embodiments and subsets of compounds, R⁸ and R^{8a} are preferably each selected from hydrogen; C₁₋₄ alkoxy; nitro; amino; mono- and di-C₁₋₄ alkylamino; and guanidinyl.

More preferably, R⁸ and R^{8a} are each selected from hydrogen; C₁₋₄ alkoxy; amino and guanidinyl.

Still more preferably, R⁸ and R^{8a} are each selected from methoxy and amino.

In one embodiment, R⁸ and R^{8a} are both amino.

In another embodiment, R⁸ and R^{8a} are both methoxy.

In another embodiment, R⁸ and R^{8a} are both guanidinyl.

In each of the foregoing embodiments and subsets of compounds, preferably:

R⁹ is hydrogen;
R^{9a} is hydrogen;
R¹⁰ is selected from hydrogen; amino; and C₁₋₄ alkyl optionally substituted with one or more fluorine atoms;
R^{10a} is selected from hydrogen; amino; and C₁₋₄ alkyl optionally substituted with one or more fluorine atoms;
or R⁹ and R¹⁰ link together to form an alkylene chain (CH₂)_{w} wherein w is 3 to 5: and/or
R^{9a} and R^{10a} link together to form an alkylene chain (CH₂)_{w} wherein w is 3 to 5.

More preferably:

R⁹ is hydrogen;
R^{9a} is hydrogen;
R¹⁰ is selected from hydrogen; amino; methyl and trifluoromethyl;
R^{10a} is selected from hydrogen; amino; methyl and trifluoromethyl;
or R⁹ and R¹⁰ link together to form an alkylene chain (CH₂)_{w} wherein w is 3 to 5: and/or
R^{9a} and R^{10a} link together to form an alkylene chain (CH₂)_{w} wherein w is 3 to 5.

In one particularly preferred group of compounds within formula (IV):

A is a bond;
the sum of p and q is in the range from 8 to 18;
R⁸ and R^{8a} are each selected from hydrogen; C₁₋₄ alkoxy; amino and guanidinyl;
R⁹ is hydrogen;
R^{9a} is hydrogen;
R¹⁰ is selected from hydrogen; amino; and C₁₋₄ alkyl optionally substituted with one or more fluorine atoms;
R^{10a} is selected from hydrogen; amino; and C₁₋₄ alkyl optionally substituted with one or more fluorine atoms;
or R⁹ and R¹⁰ link together to form an alkylene chain (CH₂)_{w} wherein w is 3 to 5: and/or
R^{9a} and R^{10a} link together to form an alkylene chain (CH₂)_{w} wherein w is 3 to 5.

In another particularly preferred group of compounds within formula (IV):

A is a bond;
the sum of p and q is in the range from 8 to 18;
R⁸ and R^{8a} are each guanidinyl;
R⁹ is hydrogen;
R^{9a} is hydrogen;
R¹⁰ is selected from hydrogen; amino; and C₁₋₄ alkyl optionally substituted with one or more fluorine atoms;
R^{10a} is selected from hydrogen; amino; and C₁₋₄ alkyl optionally substituted with one or more fluorine atoms;
or R⁹ and R¹⁰ link together to form an alkylene chain (CH₂)_{w} wherein w is 3 to 5: and/or
R^{9a} and R^{10a} link together to form an alkylene chain (CH₂)_{w} wherein w is 3 to 5.

In another particularly preferred group of compounds within formula (IV):

A is a bond;
the sum of p and q is in the range from 8 to 18;
R⁸ and R^{8a} are each selected from hydrogen; C₁₋₄ alkoxy and amino;
R⁹ is hydrogen;
R^{9a} is hydrogen;
R¹⁰ is selected from hydrogen; amino; and C₁₋₄ alkyl optionally substituted with one or more fluorine atoms;
R^{10a} is selected from hydrogen; amino; and C₁₋₄ alkyl optionally substituted with one or more fluorine atoms;
or R⁹ and R¹⁰ link together to form an alkylene chain (CH₂)_{w} wherein w is 3 to 5: and/or
R^{9a} and R^{10a} link together to form an alkylene chain (CH₂), wherein w is 3 to 5.

Within this group, more preferred compounds are those in which:

A is a bond;
the sum of p and q is in the range from 8 to 18;
R⁸ and R^{8a} are each selected from hydrogen; methoxy and amino;
R⁹ is hydrogen;
R^{9a} is hydrogen;
R¹⁰ is selected from hydrogen; amino; methyl; and trifluoromethyl;
R^{10a} is selected from hydrogen; amino; methyl; and trifluoromethyl;
or R⁹ and R¹⁰ link together to form an alkylene chain (CH₂)_{w} wherein w is 3 to 5: and/or
R^{9a} and R^{10a} link together to form an alkylene chain (CH₂)_{w} wherein w is 3 to 5.

Within formula (IV), one preferred group of compounds may be represented by the formula (V):

[chemical structure]

wherein:
r is an integer from 2 to 24;
R⁸, R⁹ and R¹⁰ are the same or different and are each selected from hydrogen; C₁₋₄ alkyl optionally substituted with one or more fluorine atoms: C₁₋₄ alkoxy optionally substituted with one or more fluorine atoms; nitro; amino; mono- and di-C₁₋₄ alkylamino; halogen, phenyl-C₁₋₂ alkyl wherein the phenyl moiety is optionally substituted with one or two methoxy, methyl or halogen substituents; ureido and guanidinyl; or R⁹ and R¹⁰ link together to form an alkylene chain (CH₂)_{w} wherein w is 3 to 5; and R^{8a}, R^{9a} and R^{10a} are the same or different and are each selected from hydrogen; C₁₋₄ alkyl optionally substituted with one or more fluorine atoms: C₁₋₄ alkoxy optionally substituted with one or more fluorine atoms; nitro; amino; mono- and di-C₁₋₄ alkylamino; halogen, phenyl-C₁₋₂ alkyl wherein the phenyl moiety is optionally substituted with one or two methoxy, methyl or halogen substituents; ureido and guanidinyl; or R^{9a} and R^{10a} link together to form an alkylene chain (CH₂)_{w} wherein w is 3 to 5;
provided that:
(i) when R¹⁰ and R^{10a} are both hydrogen or are both methyl, and R⁹ and R^{9a} are both hydrogen, then at least one of R⁸ and R^{8a} is other than hydrogen, amino or dimethylamino; and
(ii) when R⁹ and R¹⁰ link together to form an alkylene chain (CH₂)_{w} wherein w is 4 and R^{9a} and R^{10a} link together to form an alkylene chain (CH₂)_{w} wherein w is 4, then at least one of R⁸ and R^{8a} is other than amino.

Preferably, r is an integer from 8 to 20, more preferably 10 to 18, for example any one of 10, 12, 13, 14, 15, 16, 17 and 18.

In one group of compounds within formula (V), R⁸ and R^{8a} are selected from methoxy and guanidinyl. Within this group of compounds, R⁹ and R^{9a} typically are both hydrogen and R¹⁰ and R^{10a} typically are selected from hydrogen, methyl, trifluoromethyl and amino.

In another group of compounds within formula (V), R⁸ and R^{8a} are selected from hydrogen, amino, mono- and di-C₁₋₄ alkylamino; methoxy and guanidinyl; R⁹ and R^{9a} are both hydrogen and R¹⁰ and R^{10a} are both trifluoromethyl.

For example, the analogue may be 10,10′-(decane-1,10-diyl)bis(9-amino-1,2,3,4-tetrahydroacridinium)dichloride (FIG. 1). This compound is referred to in this specification as Compound 7 because of its original notation in Galanakis et al. (J. Med. Chem. (1995) 35: 3536-3546) where it was being investigated as a potential inhibitor of calcium channels. The compound has subsequently been tested as an agent for delivery of DNA therapeutics to mitochondria (Weissig et al. (2001) S. T. P. Pharma Sci. 11: 91-96) in which publication the advantageous stability of the complexes formed by the Compound 7 analogue were reported. A further paper (Weissig et al. (2006) J. Liposome Res. 16: 249-264) reported that the Compound 7 analogue has lower toxicity in vitro compared to dequalinium.

As analogues of dequalinium with longer alkyl chains showed even lower toxicity, analogues with different chain lengths were considered (Weissig et al. (2006) J. Liposome Res. 16: 249-264). Thus, preferably, the alkyl chain of the dequalinium between 8 and 14 methyl groups, but with examples of chains containing as few as 3 methyl groups (Galankis et al (1996) J. Med. Chem. 39: 3592-3595) and other lipophilic cations containing as many as 36 methyl groups in the alkyl chain (Eaton et al. (2000) Angew. Chem. Int. Ed. 39: 4063-4067). More preferably, the alkyl chain has 10 or 12 methyl groups. As a result, an analogue of Compound 7 was designed with a 12 methyl groups in the alkyl chain, referred to herein after as Compound 7_{—}12 (10,10′-(dodecane-1,12-diyl)bis(9-amino-1,2,3,4-tetrahydroacridinium)dichloride (FIG. 2)).

Other suitable analogues of dequalinium are:
1-decanyl-2-methyl-4-aminoquinolinium iodide
1-butyl-2-methyl-4-aminoquinolinium iodide
1,1,1-triethyl-1-(10-iododecan-1-yl)ammonium iodide
1-[1-(N,N,N-triethylammonium-1-yl)-2-methyl-4-aminoquinolinium diiodide
1,1′-(decane-1,10-diyl)bis(4-aminopyridinium)diiodide
1-(4-pentyn-1-yl)-4-aminopyridinium chloride
1,1′-(deca-4,6-diyne-1,10-diyl)bis(4-aminopyridinium)dichloride dehydrate
2,2′-N,N′-(decane-1,10-diyl)bis(2,4-diaminopyridine) [compound 8]
2,2′-N,N′-(decane-1,10-diyl)bis(2-aminopyridine)
2,2′-N,N′-(decane-1,10-diyl)bis(1-methyl-2-aminopyridinium)diiodide
1-(4-pentyn-1-yl)-2-methyl-4-aminoquinolinium iodide
1,1′-(deca-4,6-diyne-1,10-diyl)bis(4-amino-2-methylquinolinium)diiodide hydrate
1,1′-(decane-1,10-diyl)bis(quinolinium)diiodide
1,1′-(decane-1,10-diyl)bis(9-amino-1,2,3,4-tetra-hydroacridinium)dibromide hydrate
2,2′-(decane-1,10-diyl)bis(quinoline)
2,2′-(decane-1,10-diyl)bis(1-methylquinolinium)diiodide hydrate
2,2′-(decane-1,10-diyl)bis(4-methoxyquinoline)
2,2′-(decane-1,10-diyl)bis(1-methyl-4-methoxyquinolinium)diiodide
2,2′-(dodecane-1,12-diyl)bis(1-methylquinolinium)diiodide
2,2′-(decane-1,10-diyl)bis(isoquinolinium)diiodide
1,1′-(decane-1,10-diyl)bis(4-bromoisoquinolinium)diiodide
1,1′-(decane-1,10-diyl)bis(1H-benzimidazole)
1,1′-(decane-1,10-diyl)bis(3-methylbenzimidazolium)diiodide hemihydrate
1,1′-(decane-1,10-diyl)bis(2-methylbenzimidazole)
1,1′-(decane-1,10-diyl)bis(2,3-dimethylbenzimidazolium)diiodide
1,10-bis[N-(acridin-9-yl)amino]decane dihydrochloride dihydrate
1,1′-(1,10-Decanediyl)bis[4-amino-2-methyl quinolinium]diiodide
1,1′-(1,10-Decanediyl)bis[4-aminoquinolinium]diiodide
1,1′-(1,10-Decanediyl)bis[4-N,N,dimethylaminoquinolinium]diiodide
1,1′-(1,10-Decanediyl)bis[2-methylquinolinium]diiodide
1,1′-(1,10-Decanediyl)bis[quinolinium]diiodide 1,6-Bis[N-(1-methylquinolinium-2-methyl)amino]hexane diiodide
1,1′-(1,10-Decanediyl)bis[1-amino isoquinolinium]diiodide
1,1′-(1,10-Decanediyl)bis[2-methylbenzoxazolium]diiodide
1,1′-(1,10-Decanediyl)bis[2-methylbenzothiazolium]diiodide
1,1′-(1,10-Decanediyl)bis[2-amino-1-methylbenzimidazolium]diiodide
1,1′-[(E)-5-Decene-1,10-diyl]bis[4-amino-2-methylquinolinium], diiodide
1,1′-[(Z)-5-Decene-1,10-diyl]bis[4-amino-2-methylquinolinium], diiodide
1,1′-(1,12-Dodecanediyl)bis[4-amino-2-methylquinolinium], diiodide
1,1′-(1,14-Tetradecanediyl)bis[4-amino-2-methylquinolinium], diiodide
1,1′-(1,16-Hexadecanediyl)bis[4-amino-2-methylquinolinium], diiodide
N-Decyl-4-aminoquinaldinium Iodide
1,1′-[Biphenyl-3,3′-diylbis(methylene)]-bis(4-aminoquinolinium)
Dibromide Hydrate (4), 1,1′-[Biphenyl-4,4′-diylbis(methylene)]bis(4-aminoquinolinium)Ditrifluoroacetate
1,1′-(Phenanthrene-3,6-diylbis(methylene)]bis(4-aminoquinolinium)Dibromide Hydrate Ethanoate
1,1′-[Fluorene-2,7-diylbis(methylene)]-bis(4-aminoquinolinium)Ditrifluoroacetate
1,1′-[Methylenebis(benzene-1,4-diylmethylene)]bis(4-aminoquinolinium)Dibromide Hydrate
1,1′-[Ethylenebis-(benzene-1,4-diylmethylene)]bis(4-aminoquinolinium)Dibromide Hydrate
(Z)-1,1′-[Stilbene-4,4′-diylbis(methylene)]-bis(4-aminoquinolinium)Dibromide Sesquihydrate
(E)-1,1′-[Stilbene-4,4′-diylbis(methylene)]bis-(4-aminoquinolinium)Dibromide Dihydrate
1,1′-[Ethyne-1,2-diylbis(benzene-1,4-diylmethylene)]bis(4-aminoquinolinium)Dibromide Sesquihydrate
1,1′-[Propane-1,3-diylbis(benzene-1,4-diylmethylene)]bis(4-aminoquinolinium)Dibromide Hemihydrate Ethanoate
1,1′-[Pyridine-2,6-diylbis(benzene-1,4-diylmethylene)]-bis(4-aminoquinolinium)Dibromide Hydrate
1,1′-[Butane-1,4-diylbis(benzene-1,4-diylmethylene)]bis-(4-aminoquinolinium)Dibromide Hydrate
1,1′-[1,1:4′,1″-Terphenyl-4,4″-diylbis(methylene)]bis(4-aminoquinolinium)Dibromide Trihydrate
1,1′-[Naphthalene-2,6-diyl(bis(methylene)]bis(4-aminoquinolinium)Dibromide Hydrate
1,1′-[Benzene-1,4-diylbis(methylene)]-bis(4-aminoquinolinium)Dibromide Dihydrate
1,1′-[Benzene-1,3-diylbis(methylene)]bis(4-aminoquinolinium)Dibromide Hemihydrate
1,1′-(Propane-1,3-diyl)bis(4-aminoquinolinium)diiodide
1,1′-(Butane-1,4-diyl)bis(4-aminoquinolinium)diiodide
1,1′-(Pentane-1,5-diyl)bis(4-aminoquinolinium)diiodide
1,1′-(Hexane-1,6-diyl)bis(4-aminoquinolinium)diiodide
1,1′-(Octane-1,8-diyl)bis(4-aminoquinolinium)diiodide
1,1′-(Dodecane-1,12-diyl)bis(4-aminoquinolinium)dibromide hemihydrate
1,10-Bis[N-(2-methylquinolin-4-yl)amino]decane
1,12-Bis[N-(2-methylquinolin-4-yl)amino]dodecane
1,10-Bis[(2-methylquinolin-4-yl)amino]decane
1,12-Bis[(2-methylquinolin-4-yl)amino]dodecane
1,10-Bis(N-quinolin-4-ylamino)decane
4,4′-[Decane-1,10-diylbis(oxy)]bis[quinoline]
4,4′-[Decane-1,10-diylbis(thio)]bis[quinoline]
4,4′-Dodecane-1,12-diylbis[quinoline]
1,8-Bis(N-quinolin-4-yldiamino)octane
1,8-Bis[N-(1-methylquinolinium-4-yl)amino]octane Diiodide Hydrate
1,10-Bis[N-(1-methylquinolinium-4-yl)amino]decane Diiodide
4,4′-[Decane-1,10-diylbis(oxy)]bis[1-methylquinolinium]Diiodide
4,4′-[Decane-1,10-diylbis(thio)]bis[1-methylquinolinium]Diiodide Hydrate (10).
1,1′-Dimethyl-4,4′-dodecane-1,12-diylbis[quinolinium]Diiodide
4,4′-Decane-1,10-diylbis[quinoline]
1,1′-Dimethyl-4,4′-decane-1,10-diylbis[quinolinium]Diiodide
1,10-Bis[N-(1-benzylquinolinium-4-yl)amino]decane Dibromide
1,10-Bis[N-(1-benzyl-2-methylquinolinium-4-yl)amino]-decane Bis(trifluoroacetate)
1,12-Bis[N-(1-benzyl-2-methylquinolinium-4-yl)amino]-dodecane Bis(trifluoroacetate)
1-[N-(1-Benzyl-2-methylquinolinium-4-yl)amino]-10-[N′-(2-methylquinolinium-4-yl)amino]decane Bis(trifluoroacetate)
1-[N-(1-Benzyl-2-methylquinolinium-4-yl)amino]-12-(N′-(2-methylquinolinium-4-yl)amino]dodecane Bis(trifluoroacetate)-3,5-Dimethoxybenzyl iodide
1,10-Bis[N41-(3,5-dimethoxybenzyl)-2-methylquinolinium-4-yl]amino]decane Bis(trifluoroacetate)
1-[N-[1-(3,5-Dimethoxybenzyl)-2-methylquinolinium-4-yl]amino]-10-[N′-(2-methylquinolinium-4-yl)amino]decane Bis(trifluoroacetate)
1,1′-(3-Iodopropylidene)bis[benzene]
1,10-Bis[N-[1-(3,3-diphenylprop-1-yl)-2-methylquinolinium-4-yl]amino]decane Bis(trifluoroacetate)
4,7-Dichloro-1-methylquinolinium Iodide
1,10-Bis[N-(7-chloro-1-methylquinolinium-4-yl)amino]-decane Diiodide Dihydrate
10,10′-(octane-1,8-diyl)bis(9-amino-1,2,3,4-tetrahydroacridinium)diiodide
10,10′-(dodecane-1,12-diyl)bis(9-amino-1,2,3,4-tetrahydroacridinium)diiodide
10,10′-(tetradecane-1,14-diyl)bis(9-amino-1,2,3,4-tetrahydroacridinium)diiodide
10,10′-(octadecane-1,18-diyl)bis(9-amino-1,2,3,4-tetrahydroacridinium)diiodide
5,5′-(dodecane-1,12-diyl)bis(11-amino-7,8,9,10-tetrahydro-6H-cyclohepta[b]quinolinium)diiodide
1,1′-(decane-1,10-diyl)bis(4-aminoquinolinium)diiodide
1,1′-(dodecane-1,12-diyl)bis(4-aminoquinolinium)diiodide
1,1′-(decane-1,10-diyl)bis(4-methoxyquinolinium)diiodide
1,1′-(decane-1,10-diyl)bis(2-aminoquinolinium)diiodide.

Dequalinium and its salts are commercially available, for example from (Sigma Aldrich). Methods of making suitable analogues are described in WO 97/48705, Galanakis et al. (1995) J. Med. Chem. 38: 595-606, Galanakis et al (1995) J. Med. Chem. 38: 3536-3546 and Galanakis et al (1996) J. Med. Chem. 39: 3592-3595, Abeywickrama et al. (2006) Bioorganic Medicinal Chem. 14: 7796-7803, Qin et al. (2000) J. Med. Chem. 43: 1413-1417, Campos Rosa et al (1996) J. Med. Chem. 39: 4247-4254, the contents of which are incorporated herein by reference. In particular, the synthesis of Compound 7 is described in Galanakis et al. (1995) supra and the synthesis of Compound 7_{—}12 may be derived there from.

The compounds of formula (IV) and (V) may be prepared by methods analogous to the known methods for preparing dequalinium, as described and referenced above.

For example, compounds of the formulae (IV) and (V) can be prepared by the reaction of a quinoline compound of the formula (VI):

[chemical structure]

with a compound of the formula I—(CH₂)ᵣ—I. The reaction is typically carried out at an elevated temperature, for example in the range 120° C. to 160° C., e.g. at around 150° C.

Quinoline compounds of the formula (VI) are commercially available or can be made by standard methods well known to the skilled person or methods analogous thereto, see for example Advanced Organic Chemistry by Jerry March, 4^{th} Edition, John Wiley & Sons, 1992, Organic Syntheses, Volumes 1-8, John Wiley, edited by Jeremiah P. Freeman (ISBN: 0-471-31192-8), 1995, Fiesers' Reagents for Organic Synthesis, Volumes 1-17, John Wiley, edited by Mary Fieser (ISBN: 0-471-58283-2), and Handbook of Heterocyclic Chemistry, A. R. Katritzky et al, 3^{rd} Edition, Elsevier, 2010.

Compounds of the formula (VI) wherein R⁸ is amino and R⁹ and R¹⁰ link together to form an alkylene chain (CH₂)_{w} can be prepared by means of the following reaction sequence:

[chemical structure]

The amino group may then be converted into other functional groups by standard methods, for example by Diazotisation followed by a Sandmeyer reaction.

TFDs are effective at nanomolar concentrations and have been effective at preventing growth of bacteria in vitro and in vivo at concentrations as low as 1 nM, although it is anticipated that against certain bacteria and in more complex settings, such as in a patient higher concentrations may be needed. Hence, a preferred range would therefore be between about 10 to 100 nM, and up to around 1 μM. It will be appreciated that the range encompasses concentrations in between about 10 nM and 1 μM, such as 20 nM, 20 nM, 40 nM, 50 nM, 60 nM, 70 nM, 80 nM, 90 nM, 150 nM, 200 nM, 500 nM, 750 nM, and intermediates thereof, for example 27.2 nM.

Where the delivery moiety is a compound of any one of formulae (I) to (V) such as dequalinium or an analogue thereof, complexes are formed between the nucleic acid and the compound (e.g. dequalinium or an analogue thereof using different ratios of both. The ratio is commonly referred to as the N/P ratio (for example see Zhao et al. (2007) Biomacromolecules 8: 3493-3502), which defines the number of positively charged Nitrogen atoms in the delivery molecule per negatively charged Phosphate atom in the nucleic acid, or per nucleotide when no phosphate atoms are present. Typically complexes are formed between the compound and TFDs at N/P ratios between 0.1 and 1 (which is sufficient to achieve charge neutralisation). It will be appreciated that the present invention encompasses ratios in between 0.1 and 1, such as 0.2, 0.3, 0.4, 0.5, 0.6, 0.7, 0.8, 0.9 and intermediates thereof, e.g. 0.23.

Complexes capable of transfecting bacteria in vitro are well tolerated in animal studies. Furthermore, the components of such complexes may be used at concentrations below their known Maximum Tolerated Dose (MTD). Maximum Tolerated Dose (MTD) is the highest daily dose that does not cause overt toxicity. MIC can be estimated by animal studies as the amount of compound that, when administered to a group of test animals, has no measurable affect on long term survivability. For example, administering a complex containing 1 μM of a 100 nucleotide TFD with an N/P ratio of 1 would give a dose of approximately 3 mg/kg dequalinium analogue. It will be appreciated that this dose of dequalinium analogue is substantially below the MTD of dequalinium. The analogues of dequlanium, such as Compound 7, show less in vitro toxicity than dequalinium (Weissig et al. (2006) J. Liposome Res. 16: 249-264). Because dequalinium has an MTD of 15 mg/kg in mice (Gamboa-Vujicic et al. (2006) J. Pharm. Sci. 82: 231-235) it is predicted that the complexes should be well tolerated. It is within the reasonable skill and knowledge of the skilled person to calculate and prepare suitable concentrations.

In a particularly preferred embodiment, the antibacterial complex comprises a) one or more transcription factor decoys as described above, and b) a dequalinium analogue or an analogue thereof.

Preferably, the alkyl chain of the dequalinium analogue has between 8 and 14 methyl groups, more preferably, 10 or 12 methyl groups. An example of a particularly suitable dequalinium analogue is 10,10′-(dodecane-1,12-diyl)bis(9-amino-1,2,3,4-tetrahydroacridinium)dichloride.

In a yet further aspect, the present invention residues in use of the decoy nucleic acid sequences and complexes of the invention in a suitable formulation for the treatment of C. difficile infection. Optionally, the treatment of C. difficile infection additionally comprises use of one or more antibiotics and/or other antibacterial agent(s).

In particular, the invention provides a method for treating C. difficile infection in a subject comprising administering a therapeutically effective amount of one or more decoy nucleic acid sequences or antibacterial complexes formulated as described herein to a subject in need of therapy. It will be appreciated that the subject may be administered a therapeutic amount of a single species of decoy sequence or complex, or a mixture of different decoy sequences. Where a mixture of sequences is administered, the sequences may be administered either as individual sequences or complexes. When administered as complexes, each complex may comprise a single species of decoy sequence either singly or multiple decoy sequences.

The subject may be a human or animal. The invention also provides a decoy nucleic acid sequence or antibacterial complex formulated as described herein for use in medicine, e.g. for use in treating or preventing C. difficile infection in a subject, and the use of one or more decoy nucleic acid sequences or antibacterial complexes formulated as described herein for the manufacture of a medicament for treating C. difficile infection.

The invention further relates to a pharmaceutical composition or medicament comprising one or more decoy nucleic acid sequences or one or more antibacterial complexes, at least one delivery moiety and a physiologically acceptable carrier or excipient.

Preferably, the pharmaceutical composition or medicament is for use in the treatment of C. difficile infection. As such, the or each decoy nucleic acid sequence comprises a regulator of expression of a gene or genes in C. difficile encoding one or more of:
(i) a cellular growth factor;
(ii) a cellular toxin;
(iii) a cellular sporulation factor;
(iv) a cellular stress response; or
(v) a cellular essential gene.

Preferably, the or each target transcription factor comprises a regulator of expression of one or more of:

(a) a gene or genes encoding cellular efflux pump protein, protein determining cell wall composition, cell wall density or cell wall metabolism;
(b) a cellular stress response gene or genes;
(c) a gene or genes encoding spores;
(d) a gene or genes encoding a toxin;
(e) an essential gene or genes.

Examples of suitable target transcription factors are Spo0A, TcdD, FapR and SigH.

The delivery moiety is selected from quaternary amine compounds; and bis-aminoalkanes and unsaturated derivatives thereof, wherein the amino component of the aminoalkane is an amino group forming part of a heterocyclic ring.

The composition may additionally comprise one or more antibiotic or other antibacterial compound or composition. Suitable antibiotics are described below and in WO 2009/044154 and WO 2010/038083. It will be appreciated that the lists provided therein may not be exhaustive. The present invention encompasses any suitable antibiotic or antibacterial compounds or compositions.

Examples of bactericidal antibiotics include: Vancomycin (Glycopeptide), Metronidazole (Nitromidazole), Tigecycline (Glycicycline, such as Tetracycline), Fidaxomicin (Macrocyclic), Rifaximicin (Rifampicin), Nitazoxanide (Nitrothiazol-salicylamide derivative), Ramoplanin (Glycolipodepsipeptide) and Rifalazil (Ansamycins).

The number of decoy sequences needed to show a predictable effect on expression of a targeted gene and have a bacteriostatic or bacteriocidal effect may be as little as circa 5000 molecules per cell. It has been found that as many as 1,000,000 bacterial cells are efficiently killed with as little as 1 nM of TFD (WO 2010/038083), suggesting that it is sufficient to have a transfection efficiency of less than 0.001% to achieve killing. In comparison with other nucleic acid-based strategies to tackle bacterial infections, such as antisense, this number of molecules needed to kill the cell is 100 to 1000-fold less. This partly reflects that although both antisense approaches and TFDs act to inhibit genes, TFDs act at an early step to prevent transcription whilst antisense, in the most common iteration, sterically blocks the products of transcription: many thousands of mRNAs molecules. Secondly, the TFDs have been designed to target essential genes that are positively induced, so need to be switched on for survival, and positively regulated (the transcription factor drives its own production). In vitro, this latter characteristic means that relatively few copies of the transcription factor are likely present when the gene is uninduced and so a small number of TFDs can block induction.

It may be that, in a therapeutic situation, there are more transcription factors per cell, due to natural variety amongst the bacterial population or the gene being already induced. In this situation it is expected that more TFDs will be needed to see a therapeutic effect and estimate that increasing the dose by a factor of 100 (to 100 nM) or improving the transfection efficiency (by two orders of magnitude) will be sufficient to see a beneficial effect. Transfection may be quantified using fluorescence microscopy (Zhang et al. (1996) J. Mol. Neurosci. 7: 13-28).

Pharmaceutical compositions according to the present invention, and for use in accordance with the present invention, may comprise as, or in addition to active ingredient, a pharmaceutically acceptable excipient or diluent any suitable binder, lubricant, suspending agent, coating agent, solubilising agent or other materials well known to those skilled in the art. Such materials should be non-toxic and should not interfere with the efficacy of the active ingredient. Acceptable carriers or diluents for therapeutic use are well known in the pharmaceutical art, and are described, for example, in Remington' Pharmaceutical Sciences, Mack Publishing Co. (A. R. Gennaro edit. 1985). The precise nature of the carrier or other material will depend on the route of administration, which may be oral.

The active ingredient is defined as one or more decoy nucleic acid sequences either alone or complexed (or formulated) with a delivery moiety, the delivery moiety being a quaternary derivative of quinoline or acridine. In the complex/formulation, the quaternary derivative of quinoline or acridine, such as dequalinium or its analogue, is in the form of a bolasome. The term ‘bolasome’ is used in this specification to describe vesicles of the derivative after the compound has been subjected to sonication (see Weissig and Torchilin (2001) Adv. Drug Delivery Rev. 49: 127-149)

A variety of methods may be used to deliver the decoy or antibacterial complex of the present invention to the site of bacterial infection. Methods for in vivo and/or in vitro delivery include, but are not limited to, bucchal or oral delivery, intravenous delivery, direct injection into the infection, direct exposure in aqueous or media solution, transfection (e.g. calcium phosphate, electroporation, DEAE-dextran based, and lipid mediated), transgenic expression (e.g. a decoy expression system delivered by microinjection, embryonic stem cell generation, or retroviral transfer), or any of the other commonly used nucleic acid delivery systems known in the art. Administration may be in combination with a suitable dose of antibiotic(s), with the antibiotic(s) being administered at the same time as the nucleic acid sequence, or separately.

Pharmaceutical compositions for oral administration may be in tablet, capsule, powder or liquid form. A tablet may comprise a solid carrier such as gelatine or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous injection or injection at the site of affliction, the active ingredient will be in the form of a parenterally acceptable aqueous solution which is pyrogen-free and has suitable pH, tonicity and stability. Those of relevant skill in the art are well able to prepare suitable solutions using, for example, isotonic vehicles such as sodium chloride, Ringer's injection, lactated Ringer's injection, preservatives, stabilisers, buffers, antioxidants and/or other additives may be included, as required.

For some applications, pharmaceutical formulation may not be required. For example, the antibacterial complex of the invention may be tolerated as a pharmaceutical in its own right, without the need for excipients and/or carriers.

Alternatively, the antibacterial complex may be suitable for use as an antibacterial disinfectant and so may be required in a suitable aqueous format. In which instance, the complex may further comprise aqueous and organic solvents and their combinations.

The decoy or antibacterial complex of the invention is ideally used to treat C. difficile infections of the alimenatary system, preferably the gut.

Where the decoy or antibacterial complex of the present invention is used in combination with one or more antibiotics, the antibiotic or antibacterial compound may be administered simultaneously with, or before or after the antibacterial complex of the invention. The antibiotic/antibacterial compound and decoy or antibacterial complex may be administered in the same or in separate compositions. Thus the invention includes combination therapies in which a decoy or an antibacterial complex as identified, and/or as described, herein is administered to a subject in combination with one or more antibiotics or other antibacterial therapies. The composition may additionally comprise one or more antibiotic or other antibacterial compounds or compositions.

As well as therapeutic uses, e.g. medical or veterinary, the decoys or antibacterial complexes of the present invention also have other ex vivo e.g. non-therapeutic applications, e.g. in disinfectants and cleaning products. In essence, the decoys or antibacterial complexes find use in methods where there is a need to reduce prokaryotic cell viability, kill cells, inhibit growth or reduce virulence. Thus the decoys may find use in bactericidal or bacteriostatic compositions.

Decoy nucleic acid sequences of the present invention may further be used in products that are applied to a surface, such as a work bench or hands, for a time and under conditions that are sufficient to reduce or prevent growth of a microorganism, and/or kill a microorganism, thereby reducing or preventing growth or killing a microorganism. For example, one or more decoys may be sprayed onto the surface. Such spray application is useful, for example, for preparing a surface for preparation of a foodstuff or sterilising an object to be inserted into a patient. This is because spraying the decoy formulation reduces the handling of the surface or object, thereby further reducing the risk of contamination. Hand and mouth wash applications are also contemplated within the scope of the invention.

In the above circumstances, the decoys may be formulated in a suitable aqueous format. In which instance, the formulation may comprise water to form an aqueous composition. The aqueous composition may further comprise aqueous and organic solvents and their combinations.

The invention also relates to kits for antibacterial use comprising one or more decoy nucleic acid sequences or one or more antibacterial complexes as described herein wherein the or each decoy or complex is for use in killing, inhibiting growth, reducing sporulation or reducing toxin production in C. difficile. Optionally, the kit further includes one or more antibiotics or other antibacterial agent(s). Typically the kit includes instructions for use. Again the kit may be for therapeutic use, e.g. against bacterial infection, or for non-therapeutic use, e.g. for cleaning or disinfecting.

All documents referred to herein are hereby incorporated by reference.

The invention will now be described in more detail by way of non-limiting examples with reference to the following figures in which:

FIG. 1: Chemical structure of 10,10′-(decane-1,10-diyl)bis(9-amino-1,2,3,4-tetrahydroacridinium)dichloride.

FIG. 2: Chemical structure of 10,10′-(dodecane-1,12-diyl)bis(9-amino-1,2,3,4-tetrahydroacridinium)dichloride.

FIG. 3: Nanoparticulate Spo0A TFD blocks sporulation in C. difficile.

FIG. 4: Cell growth of C. difficile cells treated with nanoparticles (NP) or untreated.

FIG. 5: Relative toxin production of C. difficile cells treated with nanoparticles or untreated over time.

FIG. 6: C. difficile cells treated with nanoparticulate FapR TFD prevent cell growth.

FIG. 7: Nanoparticles loaded with SigH TFD have a more antibacterial activity than empty.

FIG. 8: SigH TFD blocks early sporulation in C. difficile.

### BRIEF DESCRIPTION OF THE SEQUENCES

SEQ ID NO:1: consensus sequence for TcdD transcription factor binding site.

SEQ ID NO:2: variant binding site for the TcdD transcription factor.

SEQ ID NO:3: variant binding site for the TcdD transcription factor.

SEQ ID NO:4: variant binding site for the TcdD transcription factor.

SEQ ID NO:5: variant binding site for the TcdD transcription factor.

SEQ ID NO:6: Spo0A binding site.

SEQ ID NO:7: Spo0A binding site.

SEQ ID NO:8: sigma-54 consensus sequence

SEQ ID NO:9: Spo0A transcription factor binding site

SEQ ID NO:10: TcdR transcription factor binding site

SEQ ID NO:11: FapR transcription factor binding site

SEQ ID NO:12: SigH transcription factor binding site

SEQ ID NO:13: Spo0A TFD dumbbell.

SEQ ID NO:14: Spo0A TFD dumbbell.

SEQ ID NO:15: TcdR TFD dumbbell.

SEQ ID NO:16: TcdR TFD dumbbell.

SEQ ID NO:17: FapR TFD dumbbell.

SEQ ID NO:18: FapR TFD dumbbell.

SEQ ID NO:19: SigH TFD dumbbell.

SEQ ID NO:20: SigH TFD dumbbell.

### EXAMPLES

Although in general many of the techniques mentioned herein are well known in the art, reference may be made in particular to Sambrook and Russell, 3^{rd} Edition 2001, Molecular Cloning: a laboratory manual.

### Example 1

### Spo0A TFD Blocks Sporulation of C. difficile

TFDs were designed to block the binding of the transcription factor Spo0A that induces sporulation in C. difficile. The TFDs were assembled into nanoparticles capable of carrying the TFD into the cytoplasm of C. difficile, where the TFD binds to and competitively inhibits the action of Spo0A. Hence, when the nanoparticles were mixed with Spo0A TFD-laden nanoparticles, the number of spores produced was found to be dramatically reduced in comparison to a control sample.

### Materials and Methods

### Preparation of TFD Dumbbells by Ligation (DB-TFD)

Two phosphorylated oligonucleotides were synthesised, each containing one strand of the recognition site for the C. difficile Spo0A transcription factor. At either end of the molecule, a small hairpin loop acted to protect the molecule from degradation. Each oligonucleotide was re-suspended in dH₂O at a concentration of 250 pmol/μl:

SEQ ID NO: 13-Spo0ADB1:
CTT GGT TTT TCC AAG AGC TTG TGT AGA AGG TTG TCC
ACA AGT TGT GAA ATT TGT CGA AAA GCT ATT TAT C
SEQ ID NO: 14-Spo0ADB2:
CCC TCT TTT TGA GGG GAT AAA TAG CTT TTC GAC AAA
TTT CAC AAC TTG TGG ACA ACC TTC TAC ACA AGC T

When annealed, these formed the following molecule: a single stranded circle with a double-stranded middle constituting the binding site for Spo0A, flanked by two short hairpins, which is referred to as a ‘dumbbell TFD’. To achieve this, 30 μl of each oligonucleotide was mixed with 27 μl of dH₂O and annealed using the following PCR programme: ANNEAL: 95° C. 3 min, cool at −0.1° C./s to 8° C., end. Following which, 10 μl of 10×NEB Ligase buffer and 3 μl HC T4 DNA ligase (NEB) were added. The mixture was incubated overnight at 16° C. The material was then extensively digested with T7 exonuclease (NEB) to remove any unligated oligonucleotides and then recovered by two rounds of ethanol precipitation. The dumbbell TFD was re-suspended in water at a concentration of 1 mg/ml.

### Formation of Nanoparticles

The delivery compound 10,10′-(dodecane-1,12-diyl)bis(9-amino-1,2,3,4-tetrahydroacridinium)dichloride (as described in WO 2011/098829; compound 7_{—}12) was dissolved in DMSO to give a final concentration of 20 mg/ml. 20 μl of compound 7_{—}12 was mixed with 20 μl of TFD in a final volume of 500 μl 50 mM MES (pH5.6) and incubated at room temperature for 20 min, to form a 100× stock working solution.

### Performing Bioassays to Measure Sporulation

All procedures were performed under anaerobic conditions at room temperature unless otherwise stated. All media and agar plates used were placed in an anaerobic chamber at least 24 hours prior to use. A standard bioassay to measure sporulation was performed using untreated cells, those mixed with nanoparticles consisting of either the Spo0A TFD or empty nanoparticles (containing no TFDs), to determine the effect on C. difficile Strain 630 (a laboratory reference strain that has been sequenced).

An initial overnight culture was generated by mixing 100 μl of a glycerol stock of C. difficile (consisting of spore cells of frozen in 10% glycerol and stored at −20° C.) with 10 ml Robertson's Cooked Meat Medium (Robertson (1916) J. Path. Bacteria 20: 327-349). The following day, 10 ml BHIS media (Brain Heart Infusion media, (McFaddin J. F. (1985) Media for Isolation-Cultivation-Identification-Maintenance of medical Bacteria, Vol. I, Williams and Wilkins, Baltimore) supplemented with 0.1% L-cysteine) was inoculated with 1% of the culture grown in Robertson's (consisting mostly of spore cells). The following day, this inoculum had a turbid appearance with an Optical Density at 600 nm (OD₆₀₀) of in excess of 1.5 with a 1 cm path length. 100 μl of this culture was used to inoculate 10 ml of BHIS which was allowed to grow until it had reached an OD₆₀₀ of approximately 0.3. At this stage the growth is due to germination of spores and consists mainly of exponentially growing vegetative cells. This culture was then used to inoculate the experimental cultures that were performed in triplicate in 10 ml cultures in BHIS incubated at 37° C. The three conditions used were: A) untreated cells; B) treated with 1% (v/v) empty nanoparticles to give a final concentration of 8 μg/ml compound 7_{—}12; C) nanoparticles loaded with Spo0A TFD to give final concentrations of 8 μg/ml compound 7_{—}12 and 0.4 μg/ml TFD.

Aliquots were withdrawn from the cultures at TO, 24, 48, 72 and 96 h and used to calculate the total cell density and spore cell density.

Calculation of Spore Cell Density To measure the number of Colony Forming Units per ml of culture (CFU/ml) of spore cells, a 0.5 ml aliquot was withdrawn and exposed to anaerobic conditions for 25 minutes at 60° C. This heat shock step efficiently kills the vegetative cells but not the spores. A serial dilution of the treated sample was prepared in PBS buffer (Phosphate Buffered Saline) over a range of 10⁻¹ to 10⁻⁶. 10 μl of each dilution was spotted in duplication onto BHIS plates supplemented with 0.1% taurocholate (which enhances germination), the spots allowed to dry and the plates incubated for 24 h at 37° C. The number of colonies was then counted and the CFU/ml of original culture calculated.

### Calculation of Total Cell Density

To measure CFU/ml of total cells, meaning vegetative cells plus spore cells, a 0.5 ml aliquot was withdrawn and exposed to anaerobic conditions for 25 minutes at room temperature (to control for the time taken to heat shock the spore sample). The samples were then processed as described above.

### Measuring Cell Growth

Optical Density was measured at 600 nm in a standard spectrophotometer quantified cell growth.

### Results

Delivery of Spo0A TFD to cells with nanoparticles resulted in strong inhibition of sporulation. In an untreated culture, where cells had entered stationary phase (not shown), the density of total cells was 1.09×10⁸ CFU/ml compared to 2.81×10⁷ CFU/ml for spores, meaning that 25.5% of cells were spores (FIG. 3). In the treated samples, 1.78×10⁸ CFU/ml were total cells and only 1.54×10² CFU/ml are spores, accounting for less then 8.66×10⁻⁵% (FIG. 3). This approximate six-fold reduction can be considered evidence that the treatment has rendered the cells asporulate.

### Example 2

### TcdR TFD to Block Toxin Production

TFDs were designed to block the binding of the transcription factor TcdR that induces the production of the two Toxins A and B (encoded by tcdAB) in C. difficile. These were assembled into nanoparticles capable of carrying the TFD into the cytoplasm of C. difficile, where they would bind to and competitively inhibit the action of TcdR. Hence, when the nanoparticles were mixed with TcdR TFD-laden nanoparticles, the relative amount of Toxins produced was found to be dramatically reduced in comparison to a control sample.

### Materials and Methods

### Preparation of TFD Dumbbells by Ligation (DB-TFD)

Two phosphorylated oligonucleotides were synthesised, each containing one strand of the recognition site for the C. difficile TcdR transcription factor, and ligated to form the TFD dumbbell:

SEQ ID NO: 15-TcdRDB1:
CTT GGT TTT TCC AAG AAG TTT ACA AAA TTA TAT
TAG AAT AAC TTT TTA TT
SEQ ID NO: 16-TcdRDB2:
CCC TCT TTT TGA GGG AAT AAA AAG TTA TTC TAA
TAT AAT TTT GTA AAC TT

These were annealed, ligated and concentrated as described in Example 1.

### Formation of Nanoparticles

The delivery compound, compound 7_{—}12, was dissolved in DMSO to give a final concentration of 20 mg/ml. 20 μl of compound 7_{—}12 was mixed with 20 μl of TFD in a final volume of 500 μl 50 mM MES (pH5.6) and incubated at room temperature for 20 min, to form a 100× stock working solution.

### Performing Bioassays to Measure Production of Toxins

A commercially available ELISA kit (Enzyme-Linked Immunosorbent Assay), Premier™ ToxinA&B, was obtained from Meridian Bioscience Inc. All assays were performed following manufacturers instructions.

Conditions of cell growth were as described in Example 1, with care being taken to ensure that the experimental culture consisted of early-exponential vegetative cells that would enter stationary phase during the course of the experiment. Toxin production is developmentally controlled and only beings to occur as cells enter this phase.

### Measuring Cell Growth

Optical Density was measured at 600 nm in a standard spectrophotometer quantified cell growth.

### Results

Growth curves confirmed that treatment of C. difficile with nanoparticles loaded with the TcdR TFD failed to grow in a manner similar to untreated cells (FIG. 4). The absorbance of the cells was used to assay an equal amount of biomaterial in each experiment using the described ELISA kit. The read-out was a colourimetric one and the most intense colour was seen for untreated cells at 48 h. This was set at 100% activity and all other samples were compared to this (FIG. 5). Several trends were evident: the untreated control only produces significant amounts of Toxins after 48 h of incubation; the cells treated with empty nanoparticles produce a largely similar pattern of production to the untreated; those cells treated with the TcdR loaded nanoparticle produces much less of the Toxins. At 48 h, where maximal production is expected, only 22% of the untreated level of production was achieved. The maximum level of production for the treated cells was 32% at 72 h. Hence, the TcdR TFD effectively reduced levels of toxins production in vitro to such an extent that, if the results were reproduced in vivo, the symptoms of CDAD would be expected to be significantly reduced.

### Example 3

### FapR TFD Prevents C. difficile Growth In Vitro

TFDs were designed to block the binding of a transcription factor, FapR, which is significantly induced on damage to C. difficile walls by antibiotics such as amoxicillin. FapR was targeted because it was hypothesised that treatment with nanoparticles would also damage the cell wall and induce similar pathways. The function of the transcription factor was to control enzymes involved in fatty acid metabolism, themselves key for repair to the cell wall.

### Materials and Methods

### Preparation of TFD Dumbbells by Ligation (DB-TFD)

Two phosphorylated oligonucleotides were synthesised, each containing one strand of the recognition site for the C. difficile FapR transcription factor, and ligated to form the TFD dumbbell:

SEQ ID NO: 17-FapRDB1:
CTT GGT TTT TCC AAG TAG AAT TAG TAC CTG ATA
CTA ATA ATT
SEQ ID NO: 18-FapRDB2:
GCC TCT TTT TGA GGC AAT TAT TAG TAT CAG GTA
CTA ATT CTA

These were annealed, ligated and concentrated as described in Example 1.

### Formation of Nanoparticles

The delivery compound, compound 7_{—}12, was dissolved in DMSO to give a final concentration of 20 mg/ml. 20 μl of compound 7_{—}12 was mixed with 20 μl of TFD in a final volume of 500 μl 50 mM MES (pH5.6) and incubated at room temperature for 20 min, to form a 100× stock working solution.

### Measuring Cell Growth

Optical Density was measured at 420 nm in a standard spectrophotometer quantified cell growth.

### Results

Growth curves confirmed that treatment of C. difficile with empty nanoparticles and those loaded with the FapR TFD showed a marked difference (FIG. 6): nanoparticles containing FapR failed to grow over the time course of the experiment as measured by absorbance. Hence, delivery of this TFD blocks the action of the FapR transcription factor with evident reduction in the viability of the cells.

### Example 4

### Spo0A-SigH TFD Prevents Sporulation and Attenuates C. difficile Growth In Vitro

Spo0A TFDs have been shown to block sporulation in vitro (Example 1). A Spo0A binding site lies upstream of the spolIA promoter, which encodes an alternative sigma factor (type of transcription factor) that has been implicated in controlling sporulation (potentially in a Spo0A-independent manner) and also growth (Saujet et al. (2011) J. Bac. 193: 3186-3196). SigH binds to its own promoter to regulate positively its own production, so a TFD was designed to mimic the portion of this promoter that contains binding sites for both Spo0A and SigH. This TFD was tested to see whether it blocked sporulation more effectively than a Spo0A TFD alone, or perturbed cell growth. If found to be more effective, this dual TFD may constitute a potentially better therapeutic oligonucleotide treatment for CDAD than a TFD targeted to Spo0A alone.

### Materials and Methods

### Preparation of TFD Dumbbells by Ligation (DB-TFD)

Two phosphorylated oligonucleotides were synthesised, each containing one strand of the recognition site for the C. difficile SigH transcription factor, and ligated to form the TFD dumbbell:

SEQ ID NO: 19-SigH DB1:
CTT GGT TTT TCC AAG TAA TAC GAC AAA CTA GTT AAA
TTT CAT TGA AGG AAT AAA AAT ATA ATT ATA GAA TTG
ATT A
SEQ ID NO: 20-SigH DB2:
GCC TCT TTT TGA GGC TAA TCA ATT CTA TAA TTA TAT
TTT TAT TCC TTC AAT GAA ATT TAA CTA GTT TGT CGT
ATT A

These were annealed, ligated and concentrated as described in Example 1.

### Formation of Nanoparticles

The delivery compound, compound 7_{—}12, was dissolved in DMSO to give a final concentration of 20 mg/ml. 20 μl of compound 7_{—}12 was mixed with 20 μl of TFD in a final volume of 500 μl 50 mM MES (pH5.6) and incubated at room temperature for 20 min, to form a 100× stock working solution.

### Measuring Cell Growth

Growth was determined by the Minimum Inhibitory Concentration (MIC) method (Wiegand (2008) Nature Protocols 3: 163-175). Essentially, the antibiotic is diluted to the point where it is ineffective and is no longer able to prevent growth, as judged by the media remaining clear. For C. difficile, these experiments were performed in duplicate in 24-well cell plates containing 2 ml BHIS media with various concentrations of empty nanoparticles or those loaded with the SigH TFD.

### Results

Growth curves confirmed that treatment of C. difficile with empty nanoparticles and those loaded with the SigH TFD showed a marked difference (FIG. 7): nanoparticles containing SigH had MICs of 3.4 nM (with reference to the compound 7_{—}12 component) whereas the empty nanoparticle saw killing at 12.3 nM. Hence, the presence of 1 nM of SigH TFD in the loaded nanoparticle is sufficient for more potent killing compared to bacteria treated with empty nanoparticles.

Secondly, delivery of this TFD blocks the action of the FapR transcription factor with evident reduction in the viability of the cells.

Additionally the SigH TFD was also seen to block sporulation significantly, having performed a time course similar to that described in Example 1 (FIG. 8).

Thus, the single TFD has an impact on both the growth of C. difficile as well as its ability to sporulate. Suggesting treatment with this TFD would be useful in preventing the bacterial infection and would counter the recurrence of the disease, which is a major unmet clinical need.

1. A decoy nucleic acid sequence comprising a binding site for a target transcription factor, wherein the binding site is not operably linked to a gene, and wherein the transcription factor comprises a regulator of expression of a gene or genes in Clostridium difficile encoding one or more of:
(i) a cellular growth factor;
(ii) a cellular toxin;
(iii) a cellular sporulation factor;
(iv) a cellular stress response; or
(v) a cellular essential gene.

2. The decoy nucleic acid sequence as claimed in claim 1, wherein the target transcription factor comprises a regulator of expression of one or more of:
(a) a gene or genes encoding cellular efflux pump protein, protein determining cell wall composition, cell wall density or cell wall metabolism;
(b) a cellular stress response gene or genes;
(c) a gene or genes encoding spores;
(d) a gene or genes encoding a toxin;
(e) an essential gene or genes.

3. The decoy nucleic acid sequence as claimed in claim 1, wherein the target transcription factor is selected from: Spo0A, TcdD, FapR and SigH.

4. The decoy nucleic acid sequence as claimed in claim 1, wherein the decoy nucleic acid sequence comprises: circular double stranded DNA or a linear oligonucleotide; and/or at least one element of secondary structure; and/or more than one copy of the transcription factor binding site; and/or additional sequence to the binding site(s); and/or modified bases or sugars to increase nuclease resistance of the nucleic acid sequence; and/or a plasmid or plasmid library.

5. The decoy nucleic acid sequence as claimed in claim 1, wherein the decoy nucleic acid sequence comprises multiple direct repeats of the transcription factor binding site.

6. The decoy nucleic acid sequence as claimed in claim 1, wherein the decoy nucleic acid sequence comprises multiple transcription factor binding sites.

7. The decoy nucleic acid sequence as claimed in claim 1, wherein the decoy nucleic acid sequence comprises a circular dumbbell.

8. The decoy nucleic acid sequence as claimed in claim 1, wherein the decoy nucleic acid sequence comprises a plasmid and wherein the plasmid has one or more copies of a monomer sequence comprising a snare sequence, the snare sequence comprising at least one transcription factor binding site wherein the binding site is not operably linked to a gene.

9. The decoy nucleic acid sequence according to claim 1 wherein the transcription factor binding site in the decoy nucleic acid sequence comprises a sequence selected from the group comprising:
(SEQ ID NO: 9)
TAC GAC A
(SEQ ID NO: 10)
TTT ACA NNN NNN NNN NNN NNN NNC TTT TT
(SEQ ID NO: 11)
TAG AAT TAG TAC CTG ATA CTA ATA ATT
(SEQ ID NO: 12)
GAA GGA ATA NNN NNN NNN NNA TAG AAT

10. The decoy nucleic acid sequence according to claim 1 wherein the decoy sequence is selected from: SEQ ID NOS: 13-14, 15-16, 17-18 and/or 19-20.

11. An antibacterial complex comprising one or more decoy nucleic acid sequences and one or more delivery moieties, wherein the decoy nucleic acid sequences comprise a binding site for a target transcription factor, wherein the binding site is not operably linked to a gene, and wherein the transcription factor comprises a regulator of expression of a gene or genes in Clostridium difficile encoding one or more of:
(i) a cellular growth factor;
(ii) a cellular toxin;
(iii) a cellular sporulation factor;
(iv) a cellular stress response; or
(v) a cellular essential gene.

12. The antibacterial complex as claimed in claim 11, wherein one or more delivery moiety is selected from quaternary amine compounds and bis-aminoalkanes and unsaturated derivatives thereof, wherein the amino component of the aminoalkane is an amino group forming part of a heterocyclic ring.

13. The antibacterial complex as claimed in claim 11, wherein one or more delivery moiety is a quaternary derivative of quinoline or acridine.

14. The antibacterial complex as claimed in claim 11, wherein one or more delivery moiety is a bis-quinolinium

15. The antibacterial complex as claimed in claim 11, wherein one or more delivery moiety is dequalinium or an analogue thereof.

16. The antibacterial complex as claimed in claim 15, wherein the alkyl chain of the dequalinium analogue has between 8 and 14 methyl groups, or has 10 or 12 methyl groups.

17. The antibacterial complex as claimed in claim 16, wherein the dequalinium analogue is 10,10′-(dodecane-1,12-diyl)bis(9-amino-1,2,3,4-tetrahydroacridinium)dichloride.

18. The use of a decoy nucleic acid sequence for the treatment of Clostridium difficile infection, wherein the decoy nucleic acid sequence comprises a binding site for a target transcription factor, wherein the binding site is not operably linked to a gene, and wherein the transcription factor comprises a regulator of expression of a gene or genes in Clostridium difficile encoding one or more of:
(i) a cellular growth factor;
(ii) a cellular toxin;
(iii) a cellular sporulation factor;
(iv) a cellular stress response; or
(v) a cellular essential gene.

19. (canceled)

20. (canceled)

21. (canceled)

22. (canceled)

23. (canceled)

24. (canceled)

25. The use of claim 18 wherein the target transcription factor comprises a regulator of expression of one or more of:
(a) a gene or genes encoding cellular efflux pump protein, protein determining cell wall composition, cell wall density or cell wall metabolism;
(b) a cellular stress response gene or genes;
(c) a gene or genes encoding spores;
(d) a gene or genes encoding a toxin;
(e) an essential gene or genes.

26. The use of claim 18 wherein the target transcription factor is selected from: Spo0A, TcdD, FapR and SigH.


SEQUENCE LISTING (20 sequences)

SEQ ID NO: 1 - DNA, 35 bp, C. difficile
aagtttacaaaattatattagaataactttttatt

SEQ ID NO: 2 - DNA, 35 bp, C. difficile
atgtttacaaattactatcagacaatctccttatc

SEQ ID NO: 3 - DNA, 35 bp, C. difficile
aagtttacatatttatttcagacaacgtctttatt

SEQ ID NO: 4 - DNA, 35 bp, C. difficile
aagtttacataattatttaataattatgtaattgt

SEQ ID NO: 5 - DNA, 35 bp, C. difficile
gtttttacaatactttattaatataaagtttattg

SEQ ID NO: 6 - DNA, 7 bp, C. difficile
  misc_feature (3)..(3): n is a, c, g, or t
tgncgaa

SEQ ID NO: 7 - DNA, 7 bp, C. difficile
tgtcgaa

SEQ ID NO: 8 - DNA, 17 bp, C. difficile
  misc_feature (6)..(12): n is a, c, g, or t
tggcannnnnnnttgcw

SEQ ID NO: 9 - DNA, 7 bp, C. difficile
tacgaca

SEQ ID NO: 10 - DNA, 29 bp, C. difficile
  misc_feature (7)..(23): n is a, c, g, or t
tttacannnnnnnnnnnnnnnnncttttt

SEQ ID NO: 11 - DNA, 27 bp, C. difficile
tagaattagtacctgatactaataatt

SEQ ID NO: 12 - DNA, 27 bp, C. difficile
  misc_feature (10)..(20): n is a, c, g, or t
gaaggaatannnnnnnnnnnatagaat

SEQ ID NO: 13 - DNA, 70 bp, C. difficile
cttggtttttccaagagcttgtgtagaaggttgtccacaagttgtgaaatttgtcgaaaagctatttatc

SEQ ID NO: 14 - DNA, 70 bp, C. difficile
ccctctttttgaggggataaatagcttttcgacaaatttcacaacttgtggacaaccttctacacaagct

SEQ ID NO: 15 - DNA, 50 bp, C. difficile
cttggtttttccaagaagtttacaaaattatattagaataactttttatt

SEQ ID NO: 16 - DNA, 50 bp, C. difficile
ccctctttttgagggaataaaaagttattctaatataattttgtaaactt

SEQ ID NO: 17 - DNA, 42 bp, C. difficile
cttggtttttccaagtagaattagtacctgatactaataatt

SEQ ID NO: 18 - DNA, 42 bp, C. difficile
gcctctttttgaggcaattattagtatcaggtactaattcta

SEQ ID NO: 19 - DNA, 76 bp, C. difficile
cttggtttttccaagtaatacgacaaactagttaaatttcattgaaggaataaaaatataattatagaattgatta

SEQ ID NO: 20 - DNA, 76 bp, C. difficile
gcctctttttgaggctaatcaattctataattatatttttattccttcaatgaaatttaactagtttgtcgtatta